(19) United States
(12) Patent Application Publication
(10) Pub. No.: US 2008/0241066 A1
(43) Pub. Date: Oct. 2, 2008

(54) Anti-PRL-3 antibodies and methods of use thereof

(76) Inventors: Chengchao Shou, Hai-dian District (CN); Lirong Peng, Western District (CN); Xiao-Jia Chang, Lincoln, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/881,274

(22) Filed: Jul. 26, 2007

Related U.S. Application Data
(63) Continuation of application No. PCT/US06/02953, filed on Jan. 27, 2006.
(60) Provisional application No. 60/647,956, filed on Jan. 28, 2005.

(51) Int. Cl. A61K 49/00; C07K 16/30; A61K 39/385; G01N 33/574; A61P 35/00

(52) U.S. Cl. 424/9.1; 530/387.1; 530/388.1; 435/7.92; 424/174.1; 424/156.1

(57) ABSTRACT

The invention relates to antibodies or antigen-binding fragments thereof which bind PRL-3 without binding to and cross-reacting with PRL-1 or PRL-2. The invention also relates to methods of identifying and treating invasive or metastatic cancer using the anti-PRL-3 antibodies and the use of anti-PRL-3 antibodies in prognostic, preventative, diagnostic and other therapeutic methods.

### RELATED APPLICATIONS

This application is continuation of International Application No. PCT/US2006/002953, which designated the United States and was filed on Jan. 27, 2006 and published in English, which claims the benefit of U.S. Provisional Application No. 60/647,956, filed on Jan. 28, 2005. The entire teachings of the above applications are incorporated herein by reference.

### BACKGROUND OF THE INVENTION

Outcomes of cancer patients become increasingly poor as tumor cells become increasingly malignant, spreading beyond the primary tumor, invading the surrounding tissue and metastasizing to distal sites in the body. In fact, metastasis is the most destructive form of malignant tumor and is the main cause of death in cancer patients. The high mortality of patients with metastatic cancer is the result of the resistance of tumor metastases to both chemotherapy and radiotherapy, making them difficult to treat. In addition, metastases are often only detected after they have become well-established at other sites, also making their treatment more challenging. Thus, the identification of markers for the increasingly aggressive and malignant forms of cancer, like those that are invasive and metastatic, will help speed and enhance the diagnosis and treatment of cancer.

Protein Tyrosine Phosphatase Type IVA, member 3 (PRL-3) is a recently identified protein tyrosine phosphatase associated with tumor cell invasion and metastasis. The PRL-3 gene encodes a 22 kDa protein with a C-terminal prenylation motif. When prenylated, PRL-3 translocates from the nucleus to the cytoplasmic membrane. PRL-3 shares at least 75% sequence identity with family members PRL-1 and PRL-2. The physiological role of PRL-3 is unclear; however, overexpression of PRL-3 was found to transform human embryonic kidney cells HEK293s and enhance the invasion and metastatic properties of Chinese hamster ovary cells in mice. Another study found that PRL-3 phosphatase activity was required for the enzyme to cause tumor metastasis and angiogenesis in mice. These results strongly suggested a role for catalytically active PRL-3 in cancer progression. Even more indicative are the observations that PRL-3 expression is significantly elevated in colorectal and gastric cancer metastases, invasive gastric and breast carcinomas and in tumor cell vasculature. In contrast, comparatively little to no PRL-3 expression has been detected in primary tumor cells, making PRL-3 an excellent marker of cancer cells that have progressed to their more advanced and deadly forms.

### SUMMARY OF THE INVENTION

The present invention relates to antibodies or functional fragments thereof that bind to PRL-3, or a portion thereof, but do not detectably bind to PRL-1 or PRL-2. As used herein, the term “functional fragment” means a fragment of the antibody that specifically binds to PRL-3, or a portion thereof. Functional fragments of the antibodies of the present invention are also referred to herein as “biologically active fragments” or “antigen-binding fragments”. The anti-PRL-3 antibodies of the present invention are typically monoclonal antibodies (mAb) or functional fragments thereof. In one embodiment the antibody is mAb 3B6, or an antigen-binding fragment thereof. In another embodiment, the antibody is mAb 5D3, or an antigen-binding fragment thereof.

The present invention also relates to cells, e.g. isolated cells, that produce an antibody or antibody fragment of the present invention, in particular, those antibodies that bind mammalian PRL-3. In particular the isolated cell is murine hybridoma 3B6, deposited under CGMCC No. 1197 or murine hybridoma 5D3 deposited under CMGCC No. 1302. The present invention also encompasses methods of producing monoclonal antibodies that specifically bind to PRL-3, or a portion thereof, but do not detectably bind to PRL-1 or PRL-2.

The invention also relates to a method of detecting the presence or absence of PRL-3, or a portion thereof, in a biological sample obtained from a subject using an antibody that specifically binds PRL-3, by contacting the sample being tested with an anti-PRL-3 antibody under conditions suitable for binding of the anti-PRL-3 antibody or fragment to PRL-3. In one embodiment, the expression or presence of PRL-3, on, or in proximity of the cells, predicts the development of or indicates the existence of cancer metastases (e.g., breast, colorectal, gastric). In another embodiment, the expression or presence of PRL-3 on, or in proximity of the cells, indicates the existence of invasive carcinoma cells (e.g., gastric, breast). In yet another embodiment, the presence of PRL-3 indicates the existence of tumor cell vascularization. To assess the presence of PRL-3, in one embodiment, the monoclonal antibody that binds PRL-3 is detectably labeled or, in another embodiment, is bound by an agent that is detectably labeled. In one embodiment, the antibody is mAb 3B6 or 5D3, or an antigen-binding fragment thereof. The biological test sample can be biopsy tissue, blood, serum, saliva, urine, cerebral spinal fluid (CSF), cell lysate or a stool sample. In the biological sample, PRL-3 can be in the cell cytosol, bound to the cell membrane or in a soluble form (e.g., in blood, serum, CSF or cell lysate).

The invention further specifically relates to a method to detect PRL-3 in blood, serum or plasma using an antibody that specifically binds PRL-3 to detect the presence of PRL-3 in the sample. The presence of PRL-3 can be determined directly or indirectly. In one embodiment, the presence of PRL-3 in the sample indicates the existence of invasive cancer cells or cancer metastases. For example, the antibodies of the present invention can be used in a sandwich ELISA assay where one antibody is used to coat a solid surface (e.g., the wells of a microtiter plate); PRL-3 in a serum sample is then contacted with the antibody-coated solid surface under conditions suitable for the PRL-3 in the sample to bind to the anti-PRL-3 antibody coating the solid surface. The presence or absence of bound PRL-3 is detected with a second antibody (which can bind to a different epitope of PRL-3). Such a second antibody is typically detectably labeled.

The invention also relates to methods of detecting tumor cells in a biological sample obtained from a mammalian subject using an antibody that specifically binds PRL-3 to assess the presence of PRL-3 in the sample. In one embodiment, the presence of PRL-3 predicts the development of or indicates the existence of colorectal or gastric metastases in the subject, while in another embodiment it indicates the existence of invasive gastric or breast carcinoma cells in the subject. In yet another embodiment, the presence of PRL-3 indicates the existence of tumor cell vasculature in the subject. The presence of PRL-3 can be determined directly or indirectly. The mammalian subject can be a human, dog, cat or the like, and, in a preferred embodiment, the subject is a human.

The invention also relates to methods of detecting cancer progression by contacting a biological sample with anti-PRL-3 antibodies, or a portion thereof, whereby PRL-3 expression indicates cancer progression. In one embodiment, cancer progression is an advance in tumor stage or tumor cell metastases, invasion or angiogenesis. PRL-3 presence can be detected directly or indirectly and, in a preferred embodiment, using mAb 3B6 or 5D3.

The invention also relates to methods of detecting the metastatic progression of any cancer/tumor cells, specifically, for example, gastric or colon tumor cell metastasis to the distal organs of a mammalian subject by administering anti-PRL-3 antibodies to the subject and determining the presence of PRL-3, where the presence of PRL-3 in distal organs indicates the metastatic progression of the colon, breast or gastric tumor cells. The distal organ can be any organ of the body. In one embodiment, the distal organ is one selected from the group of lymph nodes, liver, lung peritoneum, brain, bone and ovaries.

The invention also relates to a method of determining the prognosis of survival of a mammalian subject with colon, gastric or breast cancer using an antibody that specifically binds PRL-3 by analyzing a biological test sample for PRL-3 expression and comparing that to PRL-3 expression in a suitable control. In one embodiment, the antibody is detectably labeled, in another it is bound by an agent that is detectably labeled. For example, PRL-3 expression levels can be assessed by the staining intensity or subcellular localization indicated by the detectably labeled antibody using immunohistochemistry. A suitable antibody is, for example either mAb 3B6 or 5D3. The localization of PRL-3 to the cytoplasmic membrane or an expression of PRL-3 at, or higher than a predetermined level indicates a decreased survival time for the mammalian subject.

The invention also relates to a method of preventing metastases in a mammalian subject diagnosed with cancer using an antibody that specifically binds PRL-3, or a portion thereof, and inhibits the enzymatic or biological activities of PRL-3 associated with metastasis (e.g., phosphatase activity or prenylation), preventing metastasis of the cancer. Such methods are also referred to herein as prophylactic methods. In one embodiment the antibody administered to the mammalian subject is cytolytic. In another embodiment, the antibody is conjugated to a toxic agent. In one embodiment, the antibody is administered as an adjuvant therapy and in another embodiment is administered in combination with other cancer therapies.

The invention also relates to methods of treating a mammalian patient with metastatic or invasive cancer by causing the death of a PRL-3-expressing cell using an antibody that specifically binds PRL-3, or a portion thereof. Such methods of treatment inhibit (completely or partially) the metastatic or invasive progression of the cancer, or can slow the progression of metastatic or invasive cancers. In one embodiment, the antibody administered to the mammalian subject is cytolytic. In yet another embodiment, the antibody is conjugated to a toxic agent. In one embodiment, the antibody is administered is an adjuvant therapy and in another embodiment, it is administered in combination with other cancer therapies.

The invention further relates to compositions comprising an antibody that specifically binds PRL-3, or a portion thereof, and a physiologically or pharmaceutically suitable carrier. In one embodiment the antibody is mAb 3B6 or 5D3. The composition can be used to prevent tumor metastases in a mammalian subject or treat a mammalian patient with metastatic or invasive cancer.

The present invention also relates to methods of detecting or identifying an agent (e.g., a molecule or compound that is biological, organic or inorganic) which binds to PRL-3 and inhibits (prevents or reduces) the binding of PRL-3 to a ligand. Such methods can be competitive binding assays wherein the antibodies of the present invention compete for binding PRL-3 with candidate or test agents of interest. Agents identified by these methods can modulate PRL-3 activity wherein said PRL-3 activity includes one or more of the following: binding to a target protein, binding the cell membrane, phosphatase activity, promoting cancer progression (e.g., metastasis or invasiveness) or promoting tumor vascularization or angiogenesis.

### BRIEF DESCRIPTION OF THE DRAWINGS

The foregoing and other objects, features and advantages of the invention will be apparent from the following more particular description of preferred embodiments of the invention, as illustrated in the accompanying drawings. The drawings are not necessarily to scale, emphasis instead being placed upon illustrating the principles of the invention.

FIG. 1A is the nucleotide sequence of PRL-3 (Asc No. gi:14589855) (SEQ ID No.: 1) and FIG. 1B is the amino acid sequence of PRL-3 (Asc No. gi:14589856) (SEQ ID No.: 2).

FIG. 2A is an alignment of amino acid sequences PRL-1 (SEQ ID No.: 3), PRL-2 (SEQ ID No.: 4) and PRL-3 (SEQ ID No.: 2) highlighting residues that are not shared between them and illustrating PRL-3 structural domains.

FIG. 2B is an alignment of amino acid sequences of human PRL-3 (SEQ ID NO.: 2) and mouse PRL-3 (SEQ ID No.: 5) highlighting the residues that are different between the two species.

FIG. 3 indicates the oligonucleotide primers (SEQ ID Nos.: 6-16) used for PCR amplification of PRL-1, -2 and -3 in cloning of the genes from liver metastasis tissue.

FIGS. 4A and 4B are SDS-PAGE analyses for the expression of fusion proteins GST-PRL-1, -2 and -3 in E. coli and of the purified proteins, respectively.

FIG. 5 is a graph illustrating the results of an ELISA assay demonstrating the specificity of mAb 3B6 for GST-PRL-3.

FIG. 6 is an immunoblot analysis of the specificity of mAb 3B6 for PRL-3 protein expressed in eukaryotic cells.

FIG. 7 is an immunohistochemical assay of the reactivity of mAb 3B6 with PRL-3 in colorectal cancer tissues.

FIGS. 8A-8F are photographs showing immunohistochemical staining of PRL-3 protein in normal colorectal epithelial, primary cancers and metastases.

FIG. 9 is a table showing a statistical analysis of PRL-3 expression in 36 primary colorectal cancers and their corresponding metastatic lymph nodes.

FIG. 10 is a table showing the correlations between PRL-3 protein expression and clinicopathologic factors in 88 patients with primary colorectal cancer.

FIG. 11 is a table showing univariate and multivariate analyses of 88 patients with colorectal cancer.

FIG. 12 is a graph showing the clinical outcome of patients with colorectal cancer after surgery with a 34-month follow-up period.

FIG. 13 is an immunoblot analysis of PRL-3 protein detected in the serum samples of breast cancer patients using mAb 3B6 and mAb 5D3.

FIG. 14 is a table summarizing the results of a serum-based ELISA assay from a group of 31 breast cancer patients.

FIGS. 15A-15H are photographs showing immunohistochemical staining of PRL-3 protein in invasive breast carcinoma cells at different stages (15A-15E) and breast carcinoma cells in lymph nodes (15F-15H).

FIG. 16 is a table showing correlation data between PRL-3 expression in breast cancer tissues and clinicopathological characteristics or adjuvant therapy.

FIG. 17 is a table showing the univariate analysis of the association between PRL-3 expression and the 5-year overall survival (OS) of breast cancer patients.

FIGS. 18A-18C are Kaplan-Meier plots of the survival of PRL-3 positive and negative breast cancer patients.

FIG. 19 is a table showing the multivariate analysis of independent prognostic factors and PRL-3 expression in breast cancer patients.

### DETAILED DESCRIPTION OF THE INVENTION

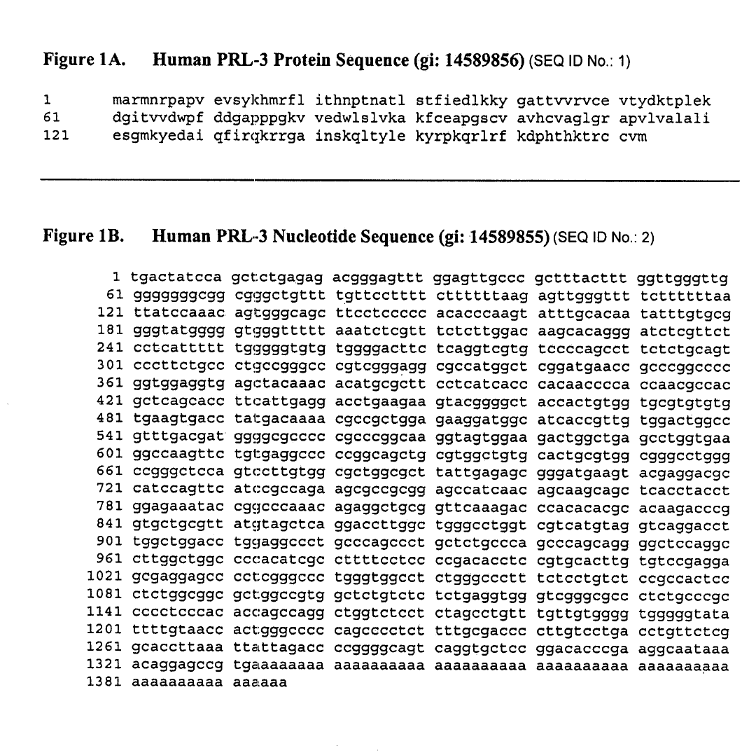

PRL-3 as used herein refers to a protein of 173 amino acids whose nucleotide and amino acid sequence are as shown in FIG. 1A (SEQ ID No.: 1) and FIG. 1B (SEQ ID No.: 2), respectively. The protein has a prenylation motif in its carboxy-terminal region. PRL-3 is a cellular and serological marker for cancer progression. “Cancer progression” or “progressive cancer” as used herein refers to an advance in tumor stage (e.g., a change from an early stage tumor to a late stage tumor) or the spread of cancer cells from the primary tumor and includes invasion—the increased motility and migration of tumor cells into nearby tissue, angiogenesis—the vascularization of tumor cells, and metastasis—the migration of tumor cells to foreign sites (i.e., distal organs) in the body. PRL-3 (the complete protein, or a fragment thereof) can be detected by histochemical or serological methods and correlated to cancer progression using the antibodies and methods as described herein.

### Antibodies and Antibody Producing Cells

The term “monoclonal antibody” or “antibody” as used herein encompasses functional fragments of antibodies, including fragments of chimeric, humanized, primatized, veneered or single chain antibodies. Functional fragments include antigen-binding fragments that bind to PRL-3. For example, antibody fragments capable of binding PRL-3 or portions thereof, including but not limited to Fv, Fab, Fab′ and F(ab′)₂ fragments are encompassed by the invention. Such fragments can be produced by enzymatic cleavage or by recombinant techniques. For example, papain or pepsin cleavage can generate Fab or F(ab′)₂ fragments respectively. Other proteases with the requisite substrate specificity can also be used to generate Fab or F(ab′)₂ fragments. Antibodies can also be produced in a variety of truncated forms using antibody genes in which one or more stop codon has been introduced upstream of the natural stop site. For example, a chimeric gene encoding a F(ab′)₂ heavy chain portion can be designed to include DNA sequences encoding the CH1 domain and hinge region of the heavy chain.

Single chain antibodies, and chimeric, humanized or primatized (CDR-grafted), or veneered antibodies, as well as chimeric, CDR-grated or veneered single chain antibodies, comprising portions derived from different species, and the like are also encompassed by the present invention and the term “monoclonal antibody” or “antibody”. The various portions of these antibodies can be joined together chemically by conventional techniques, or can be prepared as a contiguous protein using genetic engineering techniques. For example nucleic acids encoding a chimeric humanized chain can be expressed to produce a contiguous protein. See e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0,451,216 B1; and Padlan, E. A. et al., EP 0,519,596 A1.

Humanized antibodies can be produced using synthetic or recombinant DNA technology using standard methods or other suitable techniques. Nucleic acid (e.g., cDNA) sequences coding for humanized variable regions can also be constructed using PCR mutagenesis methods to alter DNA sequences encoding a human or humanized chain, such as a DNA template from a previously humanized variable region (see e.g., Kamman, M. et al., Nucl. Acids Res., 17:5404 (1989)); Sato, K., et al., Cancer Res, 53:851-856 (1993); Daugherty, B. L. et al., Nucl Acids Res, 19(9):3471-2476 (1991); and Lewis, A. P. and J. S. Crowe, Gene, 101:297-302 (1991)). Using these or other suitable methods, variants can also be readily produced. In one embodiment, cloned variable regions can be mutated, and sequences encoding variants with the desired specificity can be selected (e.g., from a phage library; see e.g., Krebger et al., U.S. Pat. No. 5,514,548; Hoogenboom et al., WO 93/06213).

Preparation of an immunizing antigen and monoclonal antibody production can be performed using any suitable technique. A variety of methods have been described (see e.g., Kohler et al., Nature, 256:495-497(1975) and Eur J Immunol 6:511-519(1976); U.S. Pat. No. 4,172,124; Harlow, E. And D. Lane, 1988, Antibodies: A Laboratory Manual, (Cold Spring Harbor Laboratory: Cold Spring Harbor, N.Y.); Current Protocols in Molecular Biology, Vol. 2 (Supplement 27, Summer '94)). Generally, a hybridoma is produced by fusing a suitable immortal cell line (e.g., a myeloma cell line) with antibody producing cells. Antibody producing cells can be obtained from the peripheral blood or, preferably the spleen or lymph nodes, of humans or other suitable animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Other suitable methods of producing or isolating antibodies of the requisite specificity (e.g., human antibodies or antigen-binding fragments) can be used, including, for example, methods which select a recombinant antibody from a library (e.g., a phage display library), or which rely upon immunization of transgenic animals (e.g., mice) capable of producing a repertoire of human antibodies (see e.g., Jakobovits et al., Proc. Natl. Acad. Sci. USA, 90:2551-2555(1993); Jakobovits et al., Nature, 362:255-258(1993); Lonberg et al., U.S. Pat. No. 5,545,806; Surani et al., U.S. Pat. No. 5,545,807; Lonberg et al., WO 97/13852).

In one embodiment, the antibody or antigen-binding fragment has specificity for PRL-3, preferably a naturally occurring or endogenous PRL-3. Specifically, the antibody or antigen-binding fragment binds to PRL-3, or a portion thereof, but does not detectably bind to PRL-1 or PRL-2. Preferably, PRL-3, or a portion thereof, is mammalian PRL-3 and, in particular, is human PRL-3. In another embodiment, the antibody is an IgG or antigen-binding fragment of an IgG.

As described herein, a monoclonal antibody designated “mAb 3B6” has been produced. mAb 3B6 can be produced by the murine hybridoma 3B6 which was deposited by Dr. Cheng-Chao Shou on Jul. 22, 2004 at the China General Microbiological Culture Collection Center, China Committee for Culture Collection of Microorganisms P.O. Box 2714, Beijing 100080, China, under CGMCC No. 1197. Another monoclonal antibody designated “mAb 5D3” has been produced. mAb 5D3 can be produced by murine hybridoma 5D3 which was deposited by Dr. Cheng-Chao Shou on Jan. 24, 2005 at the CGMCC, China Committee for Culture Collection of Microorganisms P.O. Box 2714, Beijing 100080, China, under CMGCC No. 1302.

In one embodiment, the anti-PRL-3 antibody of the invention is mAb 3B6, or an antigen-binding fragment thereof. As used herein, “antigenic site” or antibody “epitope” or “epitopic specificity” refer to the region, area or amino acids residues of the PRL-3 protein to which the antibodies of the invention bind. In another embodiment the anti-PRL-3 antibody of the invention is mAb 5D3 or an antigen-binding fragment thereof. In another embodiment, the binding of an antibody or antigen-binding fragment to PRL-3 can be inhibited by mAb 3B6 or mAb 5D3. Such inhibition can be the result of competition for the same or similar epitope or steric interference. In still another embodiment, the monoclonal antibody of the invention has the same or similar epitopic specificity as mAb 3B6 or as mAb 5D3. Antibodies with an epitopic specificity which is the same as or similar to that of mAb 3B6 or that of mAb 5D3 can be identified by a variety of suitable methods. For example, an antibody with the same or similar epitopic specificity as mAb 3B6 or mAb 5D3 can be identified based upon the ability to compete with either antibody for binding to PRL-3 or a portion of the PRL-3 protein (e.g., a PRL-3 polypeptide or peptide).

In a preferred embodiment, the antibody or antigen-binding fragment of the invention specifically binds to PRL-3. As used herein, the term “specific antibody” or “specific” when referring to an antibody-antigen interaction is used to indicate that the antibody can selectively bind to PRL-3, and does not detectably bind to PRL-1 or PRL-2. The concentration of antibody required to provide selectivity for PRL-3 (e.g., a concentration which reduces or eliminates low affinity binding) can be readily determined by suitable methods, for example, titration.

In another aspect, the invention relates to an isolated cell which produces an antibody or an antigen-binding fragment of an antibody that binds to PRL-3. In a preferred embodiment, the isolated antibody-producing cells of the invention is an immortalized cell, such as a hybridoma, heterohybridoma, lymphoblastoid cell or a recombinant cell. The antibody-producing cells of the invention have uses other than for the production of antibodies. For example, the cells of the present invention can be fused with other cells (such as suitably drug-marked human myeloma, mouse myeloma, human-mouse heteromyeloma or human lymphoblastoid cells) to produce, for example, additional hybridomas, and thus provide for the transfer of the genes encoding the antibody. In addition, the cells can be used as a source of nucleic acids encoding the anti-PRL-3 immunoglobulin chains, which can be isolated and expressed (e.g., upon transfer to other cells using any suitable technique (see e.g., Cabilly et al., U.S. Pat. No. 4,816,567; Winter, U.S. Pat. No. 4,225,539)). For instance, clones comprising a sequence encoding a rearranged anti-PRL-3 light and/or heavy chain can be isolated (e.g., by PCR) or cDNA libraries can be prepared from mRNA isolated from the cell lines, and cDNA clones encoding an anti-PRL-3 immunoglobulin chain(s) can be isolated. Thus, nucleic acids encoding the heavy and/or light chains of the antibodies or portions thereof can be obtained and used for the production of the specific immunoglobulin, immunoglobulin chain, or variants thereof (e.g., humanized immunoglobulins) in a variety of host cells or in an in vitro translation system. For example, the nucleic acids, including cDNAs, or derivatives thereof encoding variants such as a humanized immunoglobulin or immunoglobulin chain, can be placed into suitable prokaryotic or eukaryotic vectors (e.g., expression vectors) and introduced into a suitable host cell by an appropriate method (e.g., transformation, transfection, electroporation, infection), such that the nucleic acid is operably linked to one or more expression control elements (e.g., in the vector or integrated into the host cell genome), to produce a recombinant antibody-producing cell.

The antibody of the invention can be produced by a suitable method, for example, by collecting serum from an animal (e.g., mouse, human, transgenic mouse) which has been immunized with PRL-3. In another example, a suitable antibody producing cell (e.g., hybridoma, heterohybridoma, lymphoblastoid cell, recombinant cell) can be maintained, either in vitro or in vivo, under conditions suitable for expression (e.g., in the presence of inducer suitable media supplemented with appropriate salts, growth factors, antibiotics, nutritional supplements), whereby the antibody or antigen-binding fragment is produced. If desired, the antibody or antigen-binding fragment can be recovered and/or isolated (e.g., from the host cells, culture medium) and purified to the desired degree. Recovery and purification of the antibody can be achieved using suitable methods, such as, centrifugation, filtration, column chromatography (e.g., ion-exchange, gel filtration, hydrophobic-interaction, affinity), preparative native electrophoresis, precipitation and ultrafiltration. It will be appreciated that the method of production encompasses expression in a host cell of a transgenic animal (see e.g., WO 92/03918, GenPharm International).

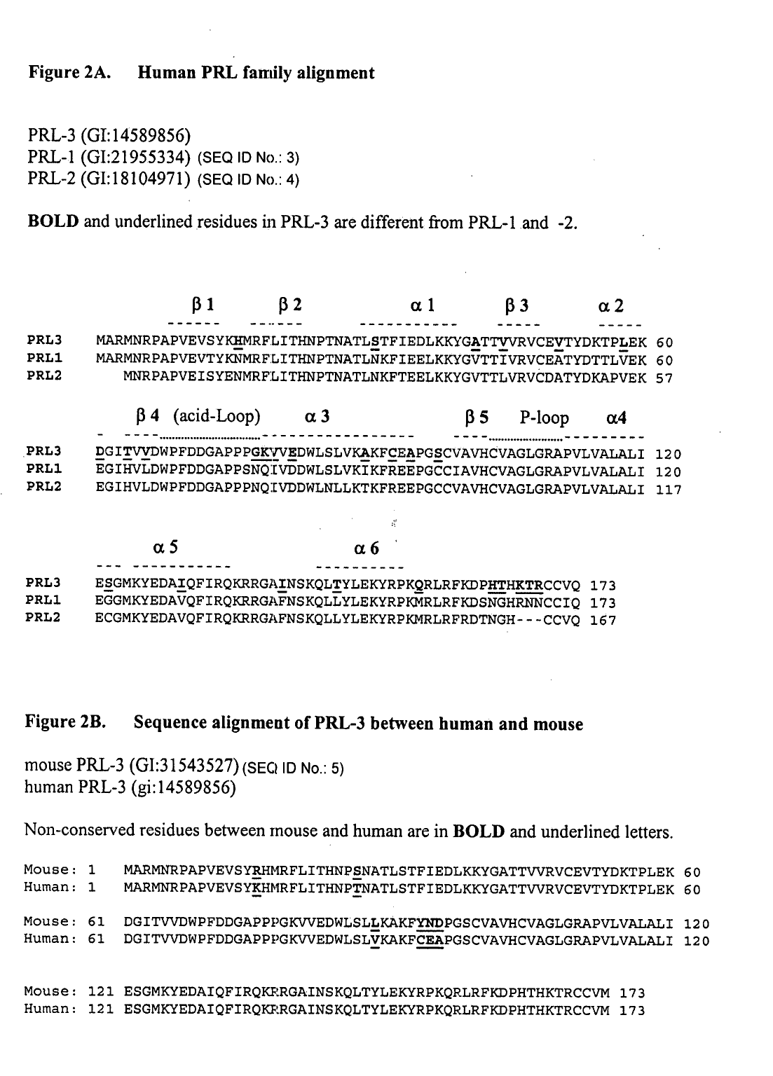

As described herein, the antibodies of the invention (e.g., polyclonal, monoclonal, functional fragments of, single chain, chimeric, humanized, or primatized) selectively and specifically bind PRL-3 without cross-reacting with PRL-1 or PRL-2. The present invention encompasses anti-PRL-3 polyclonal antibodies that do not bind to the carboxy-terminus (C-terminus) of PRL-3, in particular, the antibody of the present invention does not bind to amino acid residues 162 through 173 (see FIGS. 1 and 2A, SEQ ID No.: 1) in the C-terminal region of PRL-3. Thus, the antibodies of the invention would bind epitopes in the PRL-3 amino acid sequence that differ from PRL-1 (SEQ ID No.: 3) and PRL-2 (SEQ ID No.: 4) as shown in FIG. 2A. It is especially preferred that monoclonal antibodies of the invention specifically bind PRL-3 but not bind PRL-1 or PRL-2.

From the sequence alignment of the PRL family members and from the crystal structure of PRL-3, which illuminates residues that could be accessible to an antibody, it is reasonable to believe that the epitope sequence of mAb 3B6 or mAb 5D3 on PRL-3 may include residues like Ser31, Thr32 or Asp36 in the α 1 domain; or amino acid residues Ala42, Val45 or Val51 in the α1-β3 loop and adjacent β3 domains; or amino acid residues Gly78, Lys79, Val 80, Glu82, Ala90, Cys93 or Ala95 in the α3 domain; or amino acid residues Gln156, Pro163, His164, Thr165, Lys167, Thr168, Arg169 or Met173 in the C-terminal region of PRL-3. Of the potential sites on PRL-3 to which mAb 3B6 or mAb 5D3 may bind, it is possible at least one of those epitopes consists of the region in the PRL-3 α3 domain, as amino acid residues Val88, Cys93 and Glu94 and Ala95 also differ between mouse PRL-3 (SEQ ID No.: 5) and human PRL-3 as shown in FIG. 2B. Using, a PRL-3 polypeptide having the human amino acid sequence to immunize a mouse, for example, to produce a monoclonal antibody as described previously might make amino acid residues Val88, Cys93 and Glu94 and Ala95 more immunogenic in the mouse and thus, more likely to be an epitope site. It is likely that mAb 3B6 and mAb 5D3 bind slightly or wholly different epitopes on PRL-3. Both antibodies specifically bind PRL-3 in, for example, an ELISA assay or immunoblot; however, mAb 3B6 can also bind PRL-3 under conditions necessary to immunoprecipitate the protein from solution.

### In Vitro Diagnostic Applications

The presence or absence of a complex between PRL-3, or a portion thereof, and an anti-PRL-3 antibody (e.g., mAb 3B6 or 5D3) can be detected or determined directly or indirectly using suitable methods. For example, an antibody of the invention can be conjugated to a suitable label (e.g., a detectable label) and the formation of a complex between PRL-3 contained in the specimen and the monoclonal antibody can be determined by detection of the label. The specificity of the complex can be determined using a suitable control such as an unlabeled agent or label alone. Labels suitable for use in detection of a complex between a specimen and PRL-3 include, for example, a radioisotope, an epitope, an affinity label (e.g., biotin, avidin), a spin label, an enzyme, a fluorescent group or a chemiluminescent group. Suitable assays can be used to assess the presence or amount of PRL-3 protein such as immunological and immunochemical methods like flow cytometry (e.g., FACS analysis) and enzyme-linked immunosorbent assays (ELISA), including chemiluminescence assays, radioimmunoassay, immunoblot (e.g., Western blot) and immunohistology. Generally, a sample or specimen and the monoclonal antibody of the present invention are combined under conditions that allow the formation of an antibody-PRL-3 complex, and the complex detected as described above. In particular, the sample to be tested is contacted with an antibody of the present invention under conditions suitable for the detection of the presence or absence of PRL-3, or a portion thereof, in the sample, and the presence or absence of PRL-3 is determined. Alternatively, the antibody-PRL-3 complex can be detected by the use of a labeled agent that binds to or interacts with the complex, bound antibody or bound PRL-3. Such an agent can be, for example, a second antibody (e.g., anti-IgG if the antibody is IgG) or an antibody that binds to a second antigenic site or epitope of PRL-3. The biological specimen or sample can be, for example, a fixed slide of a tissue sample (e.g., from a tumor biopsy or other tumor tissue), a liquid sample like blood, serum, cerebral spinal fluid or urine or cell lysate. The detectable presence or absence of PRL-3 compared to a suitable control could be indicative of cancer progression.

As described herein, the expression of PRL-3, or a portion thereof, is significantly correlated with the presence of a tumor cell, and in particular, is associated with, or is a marker for, cells found in progressive cancers. Thus, the detection of the presence of PRL-3, or a portion thereof, is a marker (i.e., diagnostic tool) for the detection of the presence or absence of tumor cells, late-stage tumor cells (e.g., gastric, colorectal or ovarian), metastatic cancer cells (e.g., breast, gastric or colorectal), invasive cancer cells (e.g., gastric carcinoma or neovascular breast carcinoma) or angiogenesis associated with progressive cancer.

### In Vivo Diagnostic Applications

The antibodies of the present invention have application in determining cancer progression in procedures in which PRL-3 can be detected in tumor cells (e.g., gastric, breast, colorectal or ovarian) that have undergone cancer progression (i.e., become late-stage, invasive, metastatic or vasculogenic). The antibodies can be used to detect and/or measure the presence or expression of PRL-3 in tumor cells, or on tumor tissue or in close proximity to tumor cells. For example, the antibodies of the present invention can be administered to a mammal having, or suspected of having cancer, metastatic cancer, invasive cancer or angiogenic growth indicative of cancer, and the binding of the antibody to PRL-3, or a portion thereof can be detected in vivo directly or indirectly. The antibodies can themselves be detectably labeled or can be bound by a second agent that is detectably labeled. Such detectable labels include, for example, radioisotopes, fluorescent moieties, biotin/avidin, enzymatic labels, colorimetric labels and the like. Any label which is readily detectable using non-invasive techniques like, for instance, imaging is preferred, though invasive techniques can also be employed. In a preferred embodiment, the antibodies that specifically bind PRL-3, or a portion thereof, do not detectably bind other proteins or parts of the mammalian body, i.e., bind with low background or undetectable specific binding.

### Prognostic Applications

The antibodies of the present invention can also be used to determine the prognosis of an individual with cancer using procedures in which PRL-3, or a portion thereof, can be detected in a biological sample, such as a biopsy specimen or blood sample. The antibodies of the present invention can be used to determine the presence or expression of PRL-3 in a sample directly or indirectly using, for instance, immunohistology. For example, paraffin sections can be taken from a biopsy, fixed to a slide and combined with one or more of the antibodies of the invention by methods well-known in the art. PRL-3 expression levels can be determined by comparison to an appropriate control, for instance, immunohistology of a non-neoplastic tissue sample from an individual. PRL-3 expression higher than a predetermined threshold indicates the prognosis of survival for the individual. Similarly, tissue samples bound by the detectably labeled antibodies of the invention can be classified in stages immunohistopathologically using a scale known to those in the art (e.g., TNM-International Union Against Cancer Classification System), with a higher stage indicating a decreased prognosis for survival. For example, a classification or +3 would mean a relatively poor prognosis for the individual as compared to a classification of −1.

In addition, immunohistochemistry can be used to determine survival prognosis through PRL-3's subcellular localization. The localization of PRL-3 to the cell membrane, as opposed to the nucleus, has been associated with a more progressive form of cancer. Hence, tissue samples in which PRL-3 is bound to the cell-membrane could also indicate a decreased survival time for an individual. The localization of PRL-3 can be determined similarly to the method described above, by combining a biological sample containing cells, like a biopsy specimen or blood sample, with the antibodies of the invention and detecting the presence or absence of PRL-3 with a suitable detectable label (e.g., fluorescent or calorimetric) at the cell membrane using microscopy (e.g., light or confocal).

The measuring of PRL-3 levels in serum can be used in the management of disease. For instance, in cancer patients, serum levels of PRL-3 can be measured over time using the antibodies of the invention. PRL-3 levels can be monitored to determine a patient's response to therapies (i.e., cancer drugs). Hence, a decreased level of PRL-3 in bodily fluids, compared with the level measured prior to treatment, would indicate a positive response of the patient to a therapeutic treatment. Conversely, increasing levels of PRL-3 over time would indicate a worsening of disease progression or a resistance to therapy. Therefore, a routine monitoring of the changes in PRL-3 expression levels in those patients would be a valuable means to manage disease and guide treatment regimens.

### Therapeutic Applications

Treating cancer by killing malignant tissue has long been a standard therapy. By specifically targeting PRL-3, or a portion thereof, the antibodies of the present invention can be used therapeutically or prophylatically in procedures to cause the death of invasive or metastatic cancer cells, or to prevent or inhibit cancer progression. For example, in individuals in need of such therapy (e.g., those with a progressive cancer), an effective amount of an antibody that is cytolytic or that is coupled or conjugated to a cytotoxic agent, can be administered to the individual in order to kill or induce the apoptosis of PRL-3-expressing tumor cells. In a particularly preferred embodiment, progressive cancers that can be treated include, for instance, gastric, colorectal, breast and ovarian cancer.

According to the method, one or more agents or antibodies can be administered to the subject by an appropriate route, either alone or in combination with another drug. An effective amount of an agent (e.g., an anti-PRL-3 monoclonal antibody or antigen-binding fragment thereof) is administered. An effective amount is an amount sufficient to achieve the desired therapeutic or prophylactic effect, under the conditions of administration, such as an amount sufficient to bind PRL-3 in a cell such that it is detectable and/or such that cell death is caused. The agents can be administered in a single dose or in multiple doses to ensure the patient sustains high plasma levels of the antibody during therapy. The dosage can be determined by methods know in the art and is dependent, for example, upon the particular agent chosen, the subject's age, body weight, sensitivity and tolerance to drugs, and overall well-being. Suitable dosages for antibodies can be from about 0.01 mg/kg to about 100 mg/kg body weight per treatment.

A variety of routes of administration are possible including, for example, oral, dietary, topical, transdermal, rectal, parenteral (e.g., intravenous, intraaterial, intramuscular, subcutaneous injection, intradermal injection), and inhalation (e.g., intrabronchial, intranasal or oral inhalation, intranasal drops) routes of administration, depending on the agent and disease or condition to be treated. Administration can be local or systemic as indicated. The preferred mode of administration can vary depending on the particular agent chosen, and the particular cancer being treated; however, oral or parenteral administration is generally preferred.

The monoclonal antibodies of the invention can be administered to the individual to kill cancer cells as part of a pharmaceutical composition and a pharmaceutically acceptable carrier. Formulations will vary according to the route of administration selected (e.g., solution, emulsion, capsule). Suitable pharmaceutical carriers can contain inert ingredients which do not interact with the monoclonal antibodies. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

The assessment of PRL-3 expression levels can be used to select patients suitable for treatment with drugs (e.g., antibodies, chemicals or small molecules) that inhibit PRL-3 biological activity. The PRL-3 expression profile could be determined using the antibodies of the invention. Those patients that tested positive for PRL-3 expression would be candidates for treatment with drugs that inhibit PRL-3.

### Drug Discovery

The antibodies of the invention could also be used in methods to identify or isolate an agent (i.e., a molecule or compound) that could be used in cancer therapy as described herein. For example, it is possible that the antibodies of the invention bind PRL-3 such that its enzymatic (e.g., phosphatase) or biological (e.g., PRL-3 prenylation and translocation to the cell membrane) activity is inhibited, blocking its oncogenic function. Hence, in one embodiment, the agent is identified or isolated in a competitive binding assay in which the ability of a test agent to inhibit the binding of the antibody is assessed. The capacity of the test agent to inhibit the formation of a complex between the monoclonal antibodies of the invention and PRL-3 can be reported as the concentration of test agent required for 50% inhibition (IC50 values) or specific binding of the labeled antibodies. Specific binding is preferably defined as the total binding minus the non-specific binding. Non-specific binding is preferably defined as the amount of label still detected in complexes formed in the presence of excess unlabeled antibody. In a preferred embodiment, the antibody is mAb 3B6 or 5D3.

According to the method of the present invention, test agents can be individually screened for competitive binding or one or more agents can be tested simultaneously according to the methods herein. Where a mixture of compounds is tested, the compounds selected by the processes described can be separated (as appropriate) and identified by suitable methods (e.g., sequencing, chromatography). Test agents which bind to PRL-3 and which are useful in the therapeutic methods described herein can be identified, for example, by screening libraries or collections of molecules, such as, the Chemical Repository or the National Cancer Institute, in assays described herein or using other suitable methods. Large combinatorial libraries of compounds (e.g., organic compounds, recombinant or synthetic peptides, “peptoids”, nucleic acids) produced by combinatorial chemical synthesis or other methods can be tested (see e.g., Zuckerman, R. N. et al., J. Med. Chem., 37:2678-2685(1994); Ohlmeyer, M. H. J. et al., Proc. Natl, Acad. Sci. USA 90:10922-10926(1993), DeWitt, S. H. et al., Proc. Natl. Acad. Sci. USA 90:6909-6913(1993); Rutter, W. J. et al., U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible.

In the case that the antibodies of the invention inhibit PRL-3 enzymatic activity, they can be used to construct other agents that can be used in therapy. The antibody epitope and the three-dimensional structure or shape of the antibody epitope can be used to rationally design small molecules that have a similar shape or binding property. Such peptide mimetic can be used as therapeutic drugs, or further developed through medicinal chemical synthesis to make other organic compound drugs that block PRL-3 activity.

The present invention will now be illustrated by the following Examples, which are not intended to be limiting in any way.

### EXEMPLIFICATION

### Example 1

### Characterization of Monoclonal Antibody 3B6

### Cell Culture

Human colorectal cancer cells HT-29 were obtained from the American Type Culture Collection (ATCC, Manassas, Va.) and cultured in L-15 medium (Invitrogen Corporation, Carlsbad, Calif.) supplemented with 10% fetal bovine serum (Sigma Chemical Co., St. Louis, Mo.). Human mammary cancer cell line BICR-H1 cells and SP2/0 cells were maintained in RPMI-1640 with 20% heat-inactivated fetal calf serum (Sigma Chemical Co., St. Louis, Mo.). The cell lines were maintained in a humidified chamber with 5% CO₂ at 37° C.

Cloning of the cDNAs of PRL-1, -2, and -3

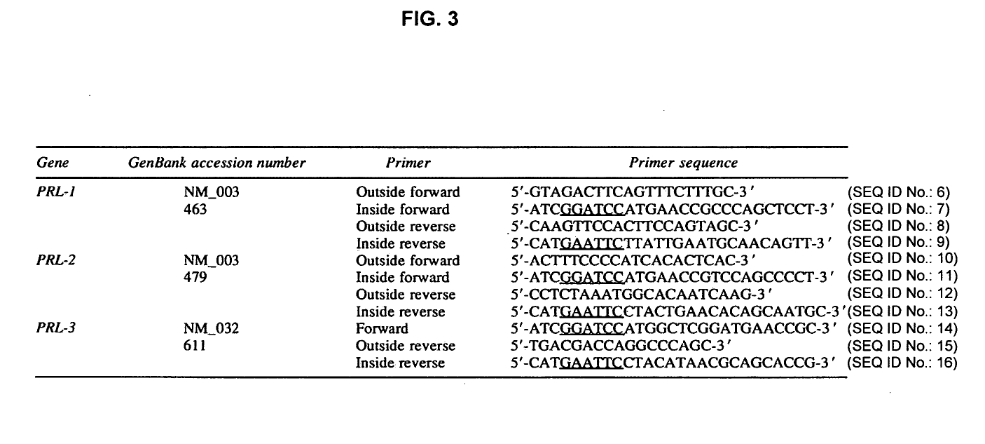

Liver metastasis tissue of colorectal cancer (approximately 0.1 g) was homogenized in Trizol reagent (Invitrogen Corporation, Carlsbad, Calif.) and total RNA was extracted according to the manufacture's protocol. For reverse transcriptase-polymerase chain reaction (RT-PCR), 10 μg of total RNA was used for PRL-3 cDNA synthesis with moloney-murine leukemia virus reverse transcriptase (M-MLV, Invitrogen Corporation, Carlsbad, Calif.) in a final volume of 50 μL. The cDNA synthesis reaction was performed at 37° C. for 1 hour, and 2 mL of reaction mixture was used to perform semi-nested PCR amplification under the following conditions: 95° C. for 5 minutes, followed by 28 cycles of 95° C. for 50 seconds, 58° C. for 50 seconds (for the outside primer) or 60° C. for 50 seconds (for the inside primer), 72° C. for 50 seconds and a final extension at 72° C. for 10 minutes. The cDNAs of PRL-1 and PRL-2 were amplified from a human embryo cDNA library by nested PCR. The oligonucleotide primers used for these amplifications (SEQ ID Nos.: 6-16) are shown in FIG. 3.

### Generation of Anti-PRL-1, -2, and -3 Antibodies

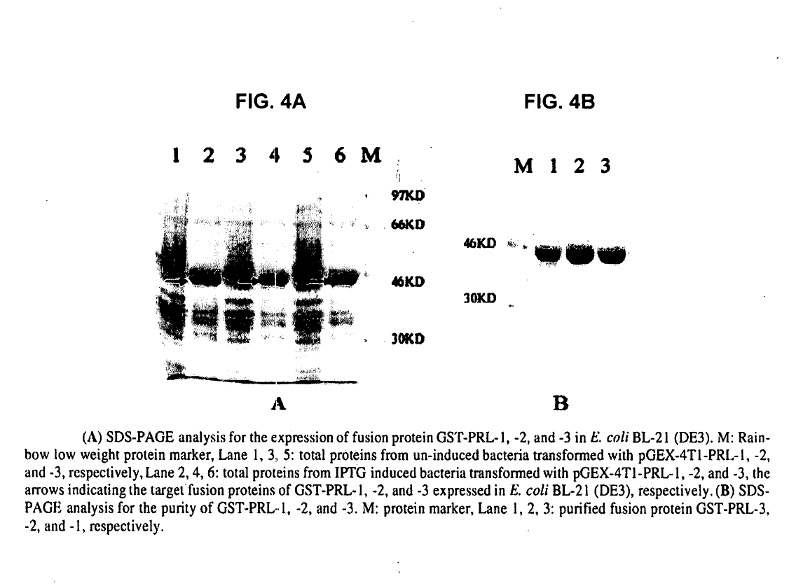

PCR fragments were digested retrieved and inserted into BamHI-EcoRI-digested pGEX-4T1 vector, respectively. Recombinant plasmids were transformed into E. coli. BL-21 (DE3) and soluble fusion proteins GST-PRL-1, -2, and -3 were produced upon the induction of 0.5 mM isopropyl-1-thi-D-galactopyranside (IPTG) at 30° C. and 250 rpm for 10 hours, respectively. The bacteria were then harvested by centrifugation and the pellets were resuspended in 5 mL of lysis buffer (1 mM PMSF, 1 mM DTT, 1 mM lysozyme, 50 mM Tris-HCl) at 4° C. for 30 minutes respectively. The mixture was sonicated on ice for six pulses and centrifuged at 12,000×g and 4° C. for 15 minutes. The supernatants containing GST-PRL-1, -2, and -3 were collected and loaded onto Glutathione Sepharose 4B affinity resin (Amersham Pharmacia Biotech, Uppsala, Sweden) pre-equilibrated with phosphate-buffered saline (PBS). After incubating overnight at 4° C., the resin was washed with PBS followed by elution buffer containing 15 mM reduced glutathione at 4° C. for 4 hours. The purities of the eluted solutions were estimated by coomassie staining after sodium dodecyl sulfate-polyacrylamide electrophoresis (SDS-PAGE) (see FIG. 4). Purified fusion proteins were used as immunogens or screening in an antigen enzyme-linked immunosorbent assay (ELISA) for the identification of monoclonal antibodies against PRL-3.

BALB/c mice (Animal Center of the National Medical Academy China) were subcutaneously immunized with 100 μg of purified GST-PRL-3 emulsified in Freund's complete adjuvant (Sigma Chemical Co., St. Louis, Mo.). Booster immunizations were carried out every 28 days with the same dosage of Freund's incomplete adjuvant and the immunological response was tested with ELISA. The immunized BALB/c mouse was given an injection of 30 μg GST-PRL-3 into the canthus vein, 3 days before cell fusion.

Hybridomas were produced by fusing spleen cells from the immunized BALB/c mouse with myeloma cell SP2/0 at a ratio of 10:1 in polyethylene glycol 4000 according to standard procedure. They were selected in RPMI-1640 medium (Invitrogen Corporation, Carlsbad, Calif.) supplemented with 20% (v/v) fetal calf serum and 5×10−3 M hypoxantine, 2×10⁻⁵ M aminopterin, and 8×10⁻⁴ M thymidine (HAT, Sigma Chemical Co.). After 10 to 15 days, supernatants of the growing hybridomas were screened for the presence of anti-PRL-3 antibodies with indirect ELISA. Selected hybridomas were subcloned four times by limiting dilution, followed by large scale preparation by developing ascetic tumors in BALB/c mice injected pristine (2, 6, 10, 14 tetramethyl-pentadecane). The isotype of the mAbs were determined using a commercial ELISA kit according to the supplier's instructions (Sigma Chemical Co., St. Louis, Mo.). The mAb was then purified with Protein A Sepharose 4B resin (Amersham Pharmacia Biotech, Uppsala, Sweden) using established methods according to the immunoglobulin isotype.

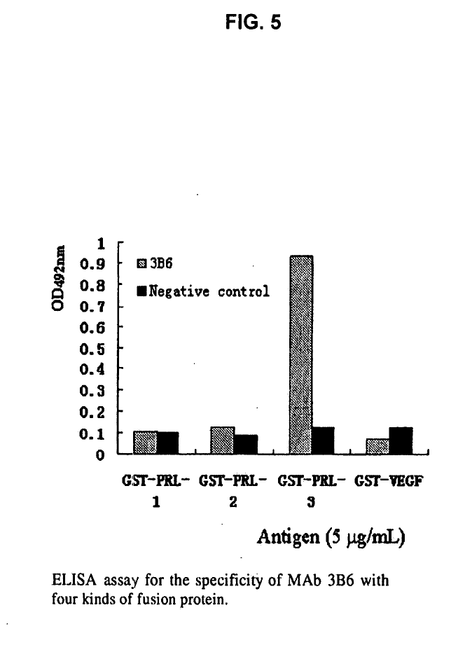

Polystyrene plates were coated with 5 μg/mL purified GST-PRL-1, -2, -3 and GST-VEGF in 0.05 M biocarbonate buffer pH 9.6 overnight at 4° C., respectively, and were washed three times with PBS containing 0.05% Tween 20. Unbound sites were blocked with 1% bovine serum alumin (BSA) at room temperature for 2 hours. The supernatants of hybridomas were added (50 μL/well) and incubated at 37° C. for 2 hours. After being washed with Tween/PBS, HRP-labeled goat anti-mouse IgG (50 μL/well, Zhongshan Corporation, Beijing, China) were added to the plates and incubated at room temperature for 1 hour. Peroxidase activity was measured with 0.5 mg/mL OPD substrate solution (100 μL/well). After 5 minutes at room temperature, the reaction was stopped by 2 M H2SO4 (50 μL/well). GST-PRL-1 and -2 were used to eliminate antibodies against PRL-1, and PRL-2; unrelated fusion protein GST-VEGF was used to eliminate antibodies against GST. One stable hybridoma designated as 3B6 was identified as IgG2a isotype and purified with protein A Sepharose 4B resin under the conditions of low salt and low pH. ELISA results showed mAb 3B6 specifically recognized PRL-3 and had no cross-reaction with PRL-1 or PRL-2 or GST (see FIG. 5).

### Analysis of mAb Binding of PRL-3

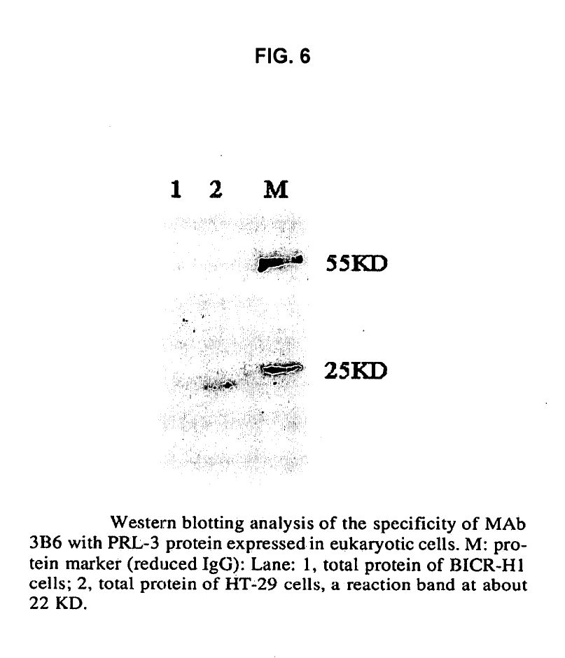

HT-29 and BICR-H1 cells were grown to confluence, washed thoroughly with PBS and detached from the monolayer with 0.25% trypsin containing 0.02% EDTA. Cells were then collected by centrifugation and lysed in SDS buffer (4% SDS, 20% glycerol, 0.12 M Tris pH 6.8, 1% bromophenol blue and 1% 2-mercaptoethanol). Lysates were immediately boiled for 10 minutes and equal amounts of protein were subjected to 12% SDS-PAGE and electroblotted to nitrocellulose paper. Non-specific binding was blocked with 5% nonfat milk in PBS overnight at 4° C. and rinsed twice with 0.1% Tween 20/PBS. Then the membrane was incubated with hybridoma supernatant at room temperature for 1 hour, followed by HRP-labeled goat anti-mouse IgG for 1 hour. The reaction product was visualized with diaminobenzidine (Sigma Chemical Co.) for 5 minutes. As shown in the western blot in FIG. 6, mAb 3B6 specifically recognized PRL-3.

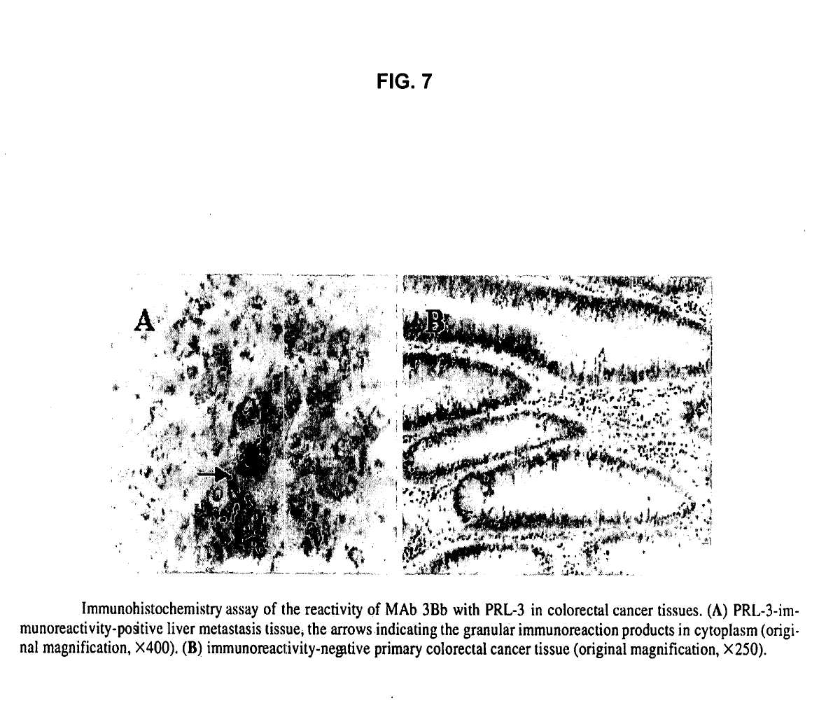

Paraffin sections (4 μm thick) of a primary colorectal cancer and liver metastases were deparaffinized with xylane and rehydrated in ethanol. Endogenous peroxidase activity was quenched by 3% hydrogen peroxide solution for 15 minutes. The sections were then blocked with 1% BSA for 1 hour and subsequently incubated with 2.5 μg/mL mAb 3B6 overnight at 4° C. Pre-immune mouse serum was used as a negative control. After being extensively washed, the sections treated with secondary antibody EnVision+™ (DAKO, Carpinteria, Calif.) for 20 minutes. The reaction was visualized with diaminobenzidine at room temperature for 1 minute. Counterstaining was performed with hematoxylin, and the sections were then hydrated and mounted. Microscopy (magnification 400× and 250×) showed immunoreactivity of PRL-3 in the cell cytoplasm but not in primary colorectal cancer tissue (see FIG. 7).

### Example 2

### Association of PRL-3 with Liver Metastasis and Colorectal Cancer Patient Prognosis

### Cancer Patients and Tissue Specimens

Tissue from 88 colorectal cancers, 28 adjacent normal colorectal epithelial (at least 5 cm distant from the tumor edge), 41 metastatic lymph nodes, and 12 liver metastases were obtained from the Department of Pathology, Peking University School of Oncology. Each sample had been fixed in formalin, routinely processed, and embedded in paraffin. Specimens were diagnosed histopathologically and staged according to the TNM-International Union Against Cancer classification system (Sobin and Witterind 1997).

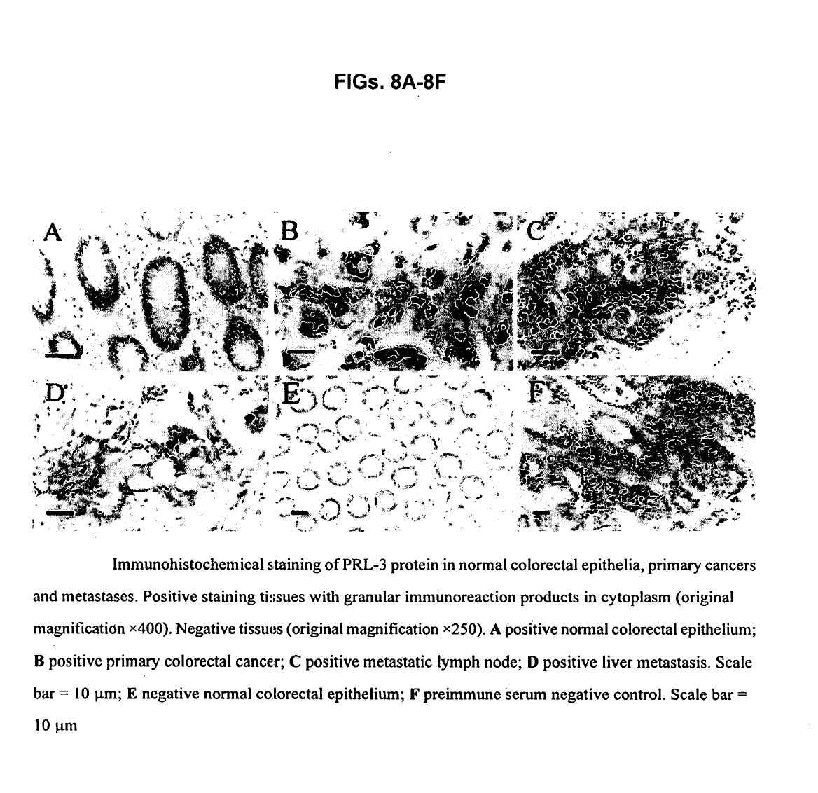

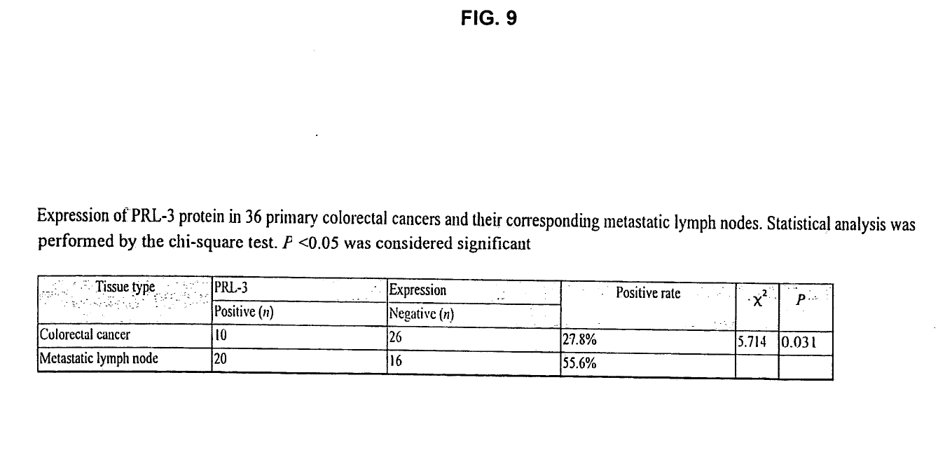

Cytoplasmic staining of colorectal cancer cells and individual normal glandular epithelial was evidenced by the presence of granular immunoreaction products (see FIG. 8A-8F). PRL-3 was expressed in 2 of 28 (7.1%) normal colorectal epithelial, 21 of 88 (23.9%) primary colorectal cancers, 22 of 41 (53.7%) metastatic lymph nodes, and 8 of 12 (66.7%) liver metastases, respectively. PRL-3 expression intensity and positive rate were not significantly different between normal colorectal epithelial and colorectal cancers, or between metastatic lymph nodes and liver metastases (P>0.05). However, PRL-3 expression rate was significantly higher in metastases when compared with normal colorectal epithelial and primary colorectal cancers (P<0.05). To more precisely determine the differences of PRL-3 protein expression in colorectal cancers, the PRL-3 expression in 36 primary colorectal cancers was compared with that in their corresponding metastatic lymph nodes. The positive rate of metastatic lymph nodes was significantly higher than that of primary colorectal cancers (see FIG. 9). Nine of ten PRL-3 immunoreactivity-positive primary cancers had positive corresponding metastatic lymph nodes. Of the 26 negative primary cancers, 11 had positive corresponding metastatic lymph nodes. The above data indicated that PRL-3 was associated with the metastasis of colorectal cancer.

### Prognostic Significance of PRL-3 Expression

Student's t-test and Pearson's x² test were performed to evaluate the possible differences between PRL-3 expression in different groups of patients, and the associations of PRL-3 expression with clinicopathologic factors. The correlation of PRL-3 with liver metastasis was studied by logistic regression. Survival analyses were calculated by the Kaplan-Meier method and Cox proportional hazard regression model. All statistical tests were two-sided and carried out with the SPSS statistical software package (SPSS 10.0, Chicago, Ill., USA). P values less than 0.05 were considered statistically significant.

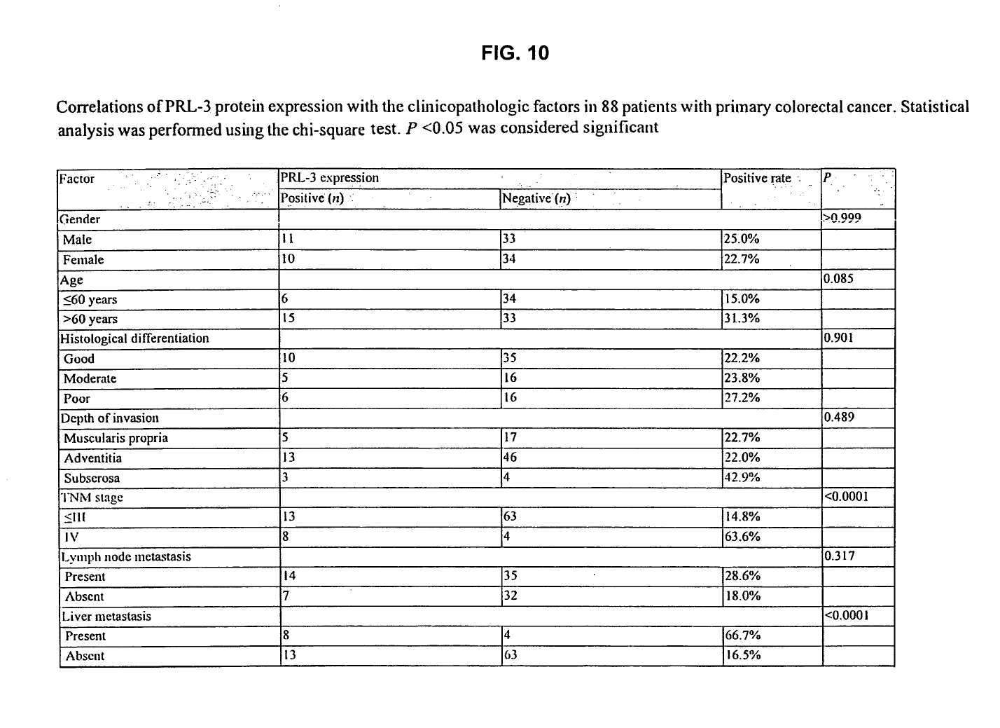

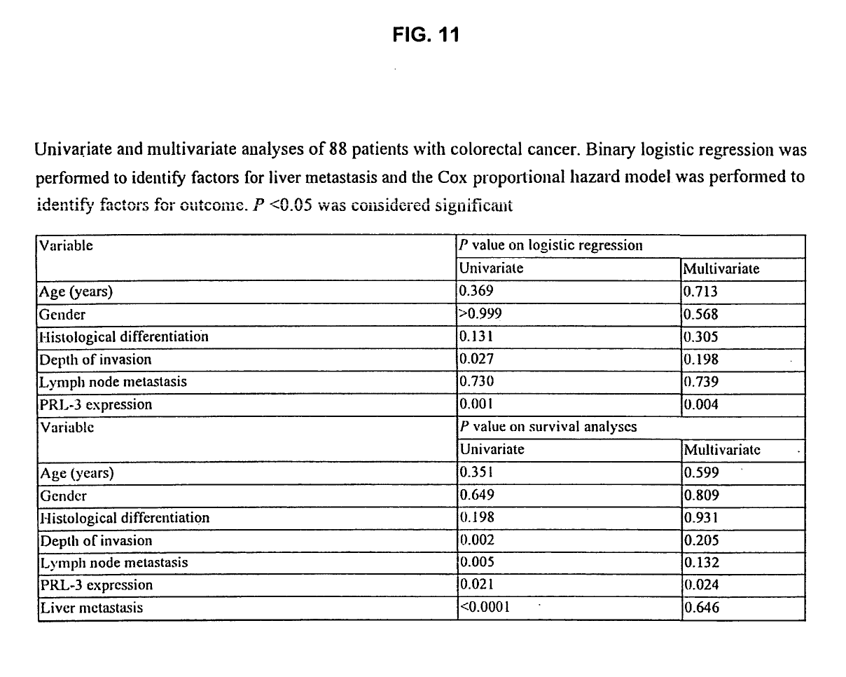

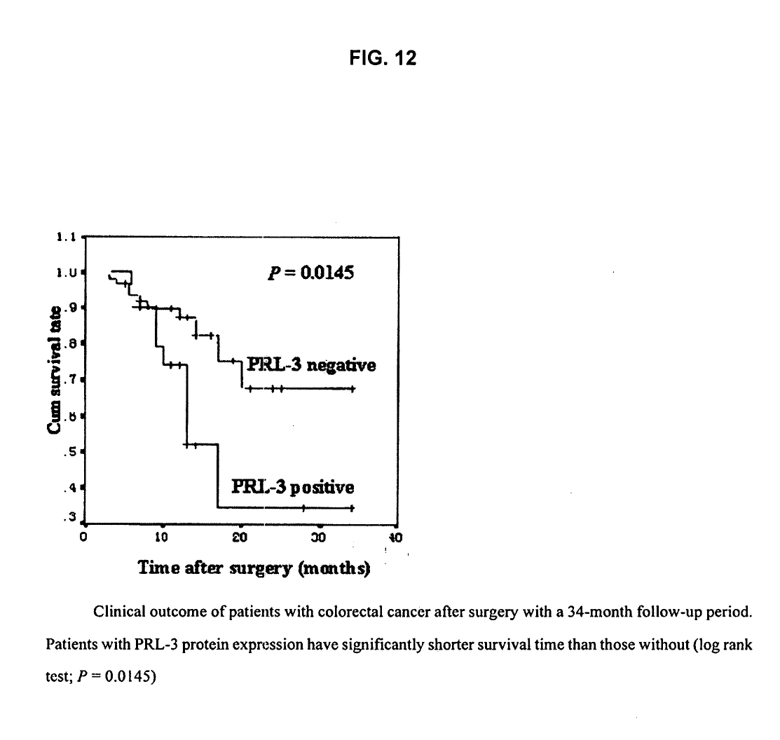

The correlations of PRL-3 immunostaining with clinicopathologic factors, which were obtained from the retrospective records of 88 patients with colorectal cancer, are summarized in FIG. 10. PRL-3 expression was positively correlated with liver metastasis (logistic regression; P=0.004). Although there were significant differences in PRL-3 expression between primary cancers and metastatic lymph nodes, the presence of lymph node metastasis could not predicted according to PRL-3 expression alone in primary cancer (FIG. 9; P>0.05), as PRL-3 positive staining in primary cancer could indicate liver metastasis (FIG. 9; P=0.004). In addition, the correlations of liver metastasis with the clinical variables of age, sex, histological differentiation, depth of invasion, lymph node metastasis, and PRL-3 expression in primary colorectal cancer were analyzed by logistic regression to identify predictors for liver metastasis. The results demonstrate that PRL-3 expression was the only independent predictor for liver metastasis (see FIG. 11). Moreover, we used the Kaplan-Meier survival curve and Cox proportional hazard model to examine the prognosis significance of PRL-3 expression for outcome. The Kaplan-Meier survival curve showed that PRL-3 expression tended to shorten the survival time of colorectal cancer patients (see FIG. 12; P=0.0145). Univariate analyses identified invasion depth, lymph node metastasis, liver metastasis, and PRL-3 positive expression as predictors for poor outcome. Multivariate analyses of these variables indicated that liver metastasis was an independent prognostic factor, but PRL-3 expression was not an independent factor.

### Discussion

A monoclonal antibody, mAb 3B6, highly specific for PRL-3, was prepared and used to determine PRL-3 protein expression in normal colorectal epithelial, primary colorectal cancers, and metastases by immunohistochemistry. PRL-3 expression rates were higher in metastatic lymph nodes and liver metastases than in primary colorectal cancers and normal epithelial, which was in agreement with the mRNA expression levels of PRL-3 reported previously (Saha S. et al., Science 294(5545):1343-1346, 2001). Statistical analyses indicated that PRL-3 expression was a predictor of liver metastasis (logistic regression; P=0.004); and patients expressing PRL-3 showed a significantly shorter survival time. A significant correlation of PRL-3 expression with lymph node metastasis was not found (P>0.05), which may be due to an insufficient number of lymph nodes excised in surgical operations or the omission of micro-lymph-node metastasis by routine histopathologic examination. This study suggested that PRL-3 immunohistochemical assessment can be used to predict liver metastasis of patients with colorectal cancer. In the future, this may provide new therapeutic strategies for treatment.

### Example 3

### Characterization of Monoclonal Antibody 5D3

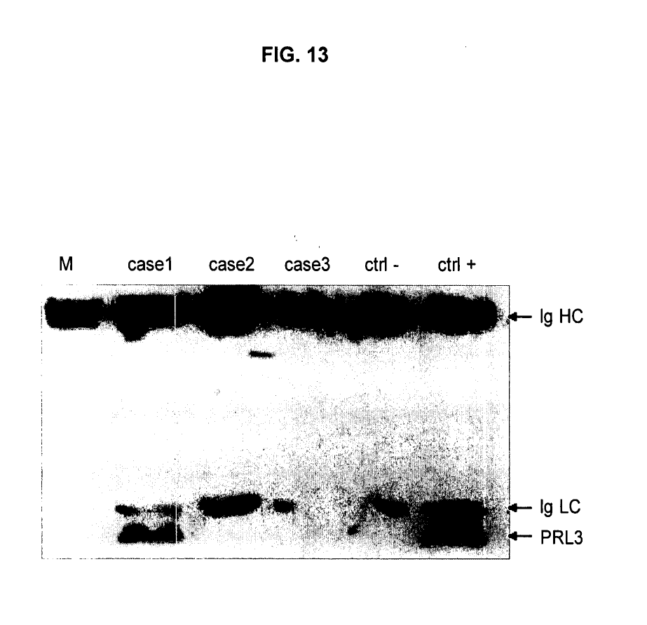

PRL-3 was immunoprecipitated from the serum samples of breast cancer patients (Specimen Bank, Beijing Cancer Research Institute) using mAb 3B6. SDS buffer (4% SDS, 20% glycerol, 0.12 M Tris pH 6.8, 1% bromophenol blue and 1% 2-mercaptoethanol) was combined with the immunoprecipitates, boiled for 10 minutes, equal amounts of protein subjected to 12% SDS-PAGE and electroblotted to nitrocellulose paper. Non-specific binding was blocked with 5% nonfat milk in PBS overnight at 4° C. and rinsed twice with 0.1% Tween 20/PBS. Then the membrane was incubated with mAb 5D3 at room temperature for 1 hour, followed by HRP-labeled goat anti-mouse IgG for 1 hour. The reaction product was visualized with diaminobenzidine (Sigma. Chemical Co.) for 5 minutes. The mAb 5D3 did not cross-react with PRL-1 or PRL-2. The immunoblot in FIG. 13 demonstrates that mAb 5D3 specifically recognized PRL-3 in COS cells transfected with PRL-3 (Lane 5) as opposed to mock-transfected cells (Lane 4). In addition, the immunoblot showed that PRL-3 was a specific marker for cancer metastases as only the breast cancer patient with bone metastasis had a PRL-3 specific band (Lanes 2-4).

### Example 4

### Serum Blood Test for PRL-3

Thirty-one serum samples of breast cancer patients were obtained (Specimen Bank, Beijing Cancer Research Institute). Polystyrene plates were coated with affinity purified 2 μg/mL rabbit polyclonal anti-PRL-3 antibody which was prepared from the anti-serum of PRL-3 immunized rabbits in 0.05 M biocarbonate buffer pH 9.6 overnight at 4° C. The plates were washed three times with PBS containing 0.05% Tween 20. Unbound sites were blocked with 1% bovine serum alumin (BSA) at room temperature for 2 hours. Serum samples diluted 1:20 were added and incubated at 37° C. for 1 hour. After being washed with Tween/PBS, mAb 3B6 at 2 μg/mL and mAb 5D3 at 6 μg/mL was added to the plates and incubated at 37° C. for 45 minutes. An HRP conjugated anti-mouse antibody was added and incubated at room temperature for 1 hour. Peroxidase activity was measured with 0.5 mg/mL OPD substrate solution (100 μL/well). After 5 minutes at room temperature, the reaction was stopped by 2 M H2SO4 (50 μL/well). Signal levels 1.8 fold or higher than that of control samples were scored as positive PRL-3 expression.

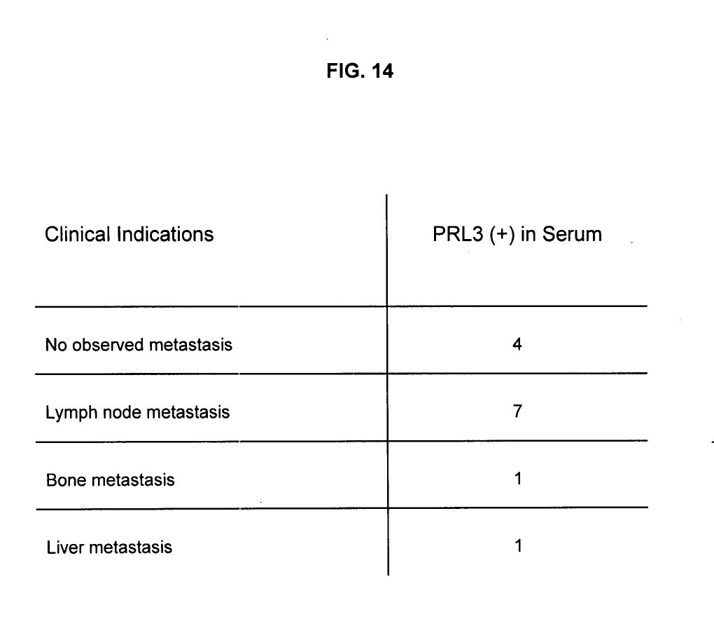

The ELISA results showed that out of the 31 serum samples, 13 were scored as PRL-3 positive. The clinical indications of breast cancer patients correlated with the PRL-3 positivity of their serum samples; the majority of those whose serum samples scored as PRL-3 positive had cancer metastases in distal sites (see FIG. 14).

### Example 5

### Prognostic Significance of PRL-3 Expression in Breast Cancer

### Study Population

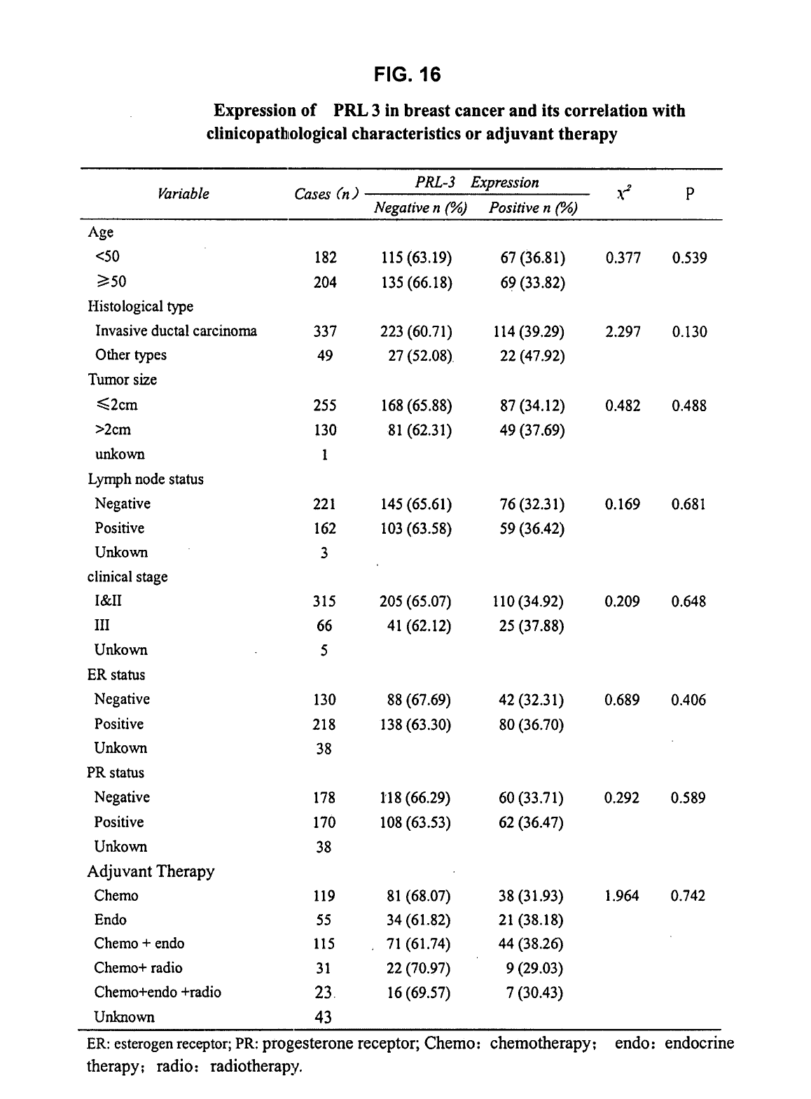

The present study enrolled 386 patients with primary breast cancer who were treated at Peking University School of Oncology between 1996 and 1999. Patients with locally recurrent tumors or tumors metastasized to the breast from other organ sites were excluded. These patients, aged from 25 to 82 (with a median of 59 years), comprised 140 perimenopausal and 246 postmenopausal patients. The stage of the tumors was classified according to the tumor-node-metastasis classification of the Union Internationale Contre Le Cancer. Patients received radical or modified radical mastectomy. The auxiliary lymph nodes were dissected to at least level I and II. Lymph node metastasis was determined based on the histological examination. The majority of patients received adjuvant therapy, including chemotherapy, endocrine therapy, radiotherapy, or combined therapy as summarized in FIG. 16. The follow-up data was available for all patients, with a median follow-up of 64 months (range 7 to 111 months). During the follow-up period, 74 (19.17%) patients died of breast carcinoma. Tumors were considered estrogen receptor or progesterone receptor positive if the specific hormone binding was ≧10 fmol/mg of cytosol protein.

### Immunohistochemistry

For immunohistochemical studies, 4 μm sections were cut from 438 paraffin blocks (386 cancer tissues, and 52 normal adjacent tissues) and baked for overnight at 50° C.-60° C. Paraffin sections were dewaxed with xylene and rehydrated through a graded alcohol series. Then, endogenous peroxidase activity was blocked in absolute methanol solution containing 3% H₂O₂ for 10 min. After being blocked by 1% BSA for 20 min, the slides were subjected to a 10-min microwave pretreatment in citrate buffer (10 mM). Then they were incubated with PRL-3 antibody mAb 3B6 (2.5 μg/ml) overnight at 4° C. in a humidified chamber. EnVision+™ (DOKO, Carpinteria, Calif.) was used as a secondary antibody. After each step, the slides were washed with PBS twice for 5 min. Antibody binding was visualized by a standard streptavid in an immunoperoxidase reaction and followed by chromagen detection with diaminobenzidine for 10 min, and hematoxylin counterstaining. Normal mouse serum was used as negative controls and the positive slides from the previous study on colon carcinoma were employed as a positive control. Staining in the cytoplasm and cytoplasmic membrane was evaluated. Samples were classified as positive when >10% of the cancer cells were stained. Immunostaining was evaluated by three oncological pathologists independently without any knowledge of the clinical data.

### Statistical Analysis

Standard x² test was performed to assess the association between PRL-3 expression and clinicopathological characteristics. Overall survival (OS) was defined as the time from diagnosis of disease to death from breast cancer or the date of last contact. Survival curves were estimated with the Kaplan-Meier method and compared by using the log rank test. Multivariate analysis was carried out by using the Cox proportional hazard regression model (a backward selection) to assess whether a factor was an independent predictor to OS. Hazard ratios (HR) with 95% confidence intervals were estimated. A two-tailed P<0.05 was considered statistically significant. All statistical analyses were performed using SPSS 10.0 software.

### Localization of PRL-3 Protein in Breast Cancer Tissues

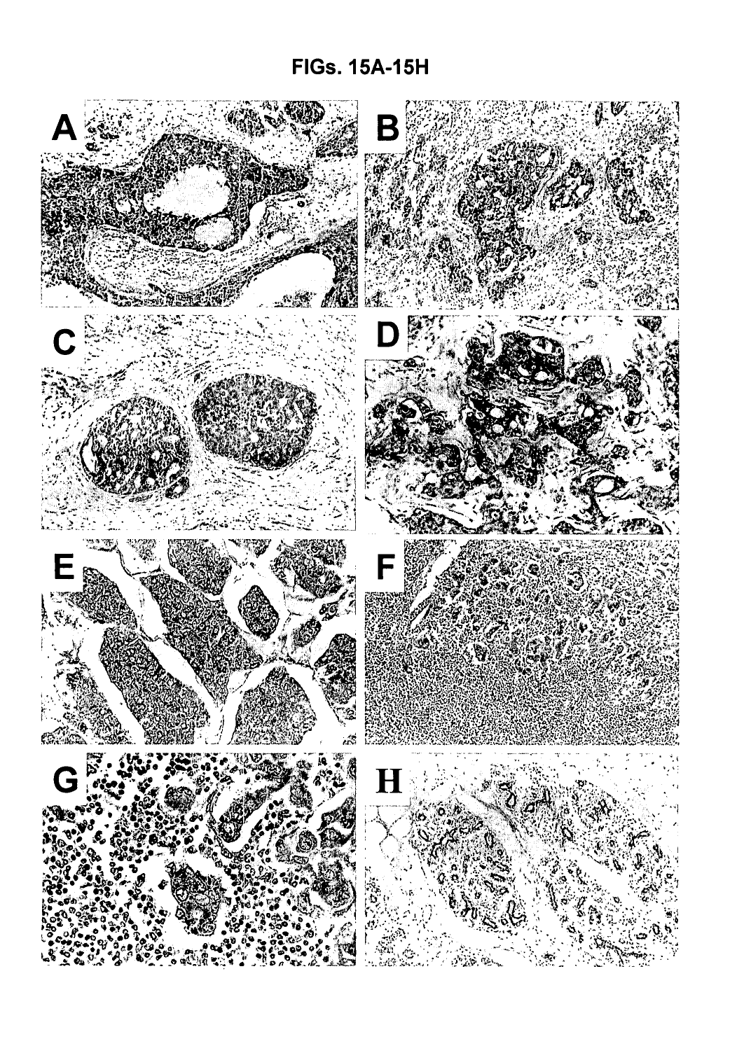

The location of PRL-3 in breast carcinoma tissues was examined by immunohistochemistry. In each case, PRL-3 was specifically localized to tumor cells. FIG. 15 (A-E) demonstrated the strong immuno-staining in invasive ductal carcinomas from patients with different clinical stage and medullary carcinoma, and PRL-3 was mainly located in the cytoplasm and the cytoplasmic membrane of the cancer cells. There was also strong PRL-3 expression in a high proportion of tumors cells in positive lymph nodes (FIGS. 15F-15H).

PRL-3 Expression in Breast Tissues and Association with Clinicopathological Characteristics or Adjuvant Therapy

Overexpression of PRL-3 was found in 136 out of 386 breast cancer tissues (35.23%), whereas only 5 out of 52 (9.62%) normal adjacent breast tissues were positive for PRL-3 expression. The difference of PRL-3 expression in the two groups was significant (x²=13.778, P<0.001). However, no correlation between PRL-3 expression (positive or negative) and various clinicopathological characteristics, such as age, histological type, tumor size, clinical stage, lymph node status, ER or PR status, was found in this cohort (see FIG. 16).

On the other hand, the adjuvant therapy, e.g., chemotherapy, endocrine therapy, radiotherapy, alone or in combination, was evenly distributed in the PRL-3 positive and negative group. No significant difference was found in these two groups (see FIG. 16). Importantly, the different clinical outcome observed in the two groups was therefore not due to the potential effects of different adjuvant therapies (see FIG. 16).

### PRL-3 Expression and Breast Cancer Survival

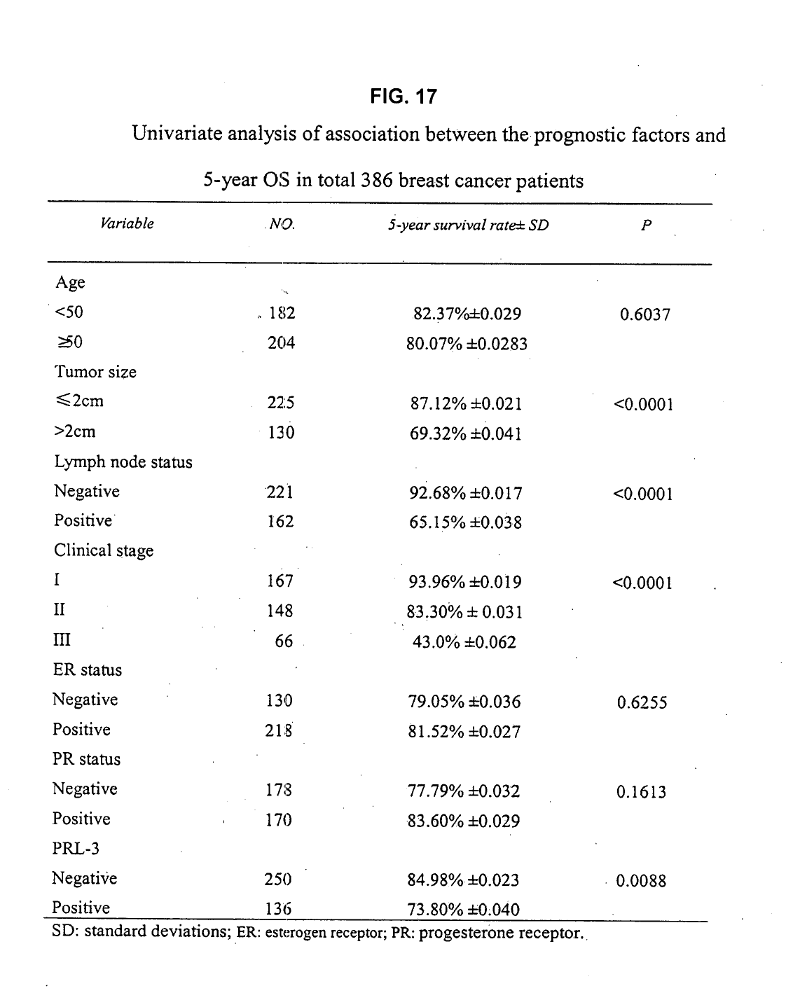

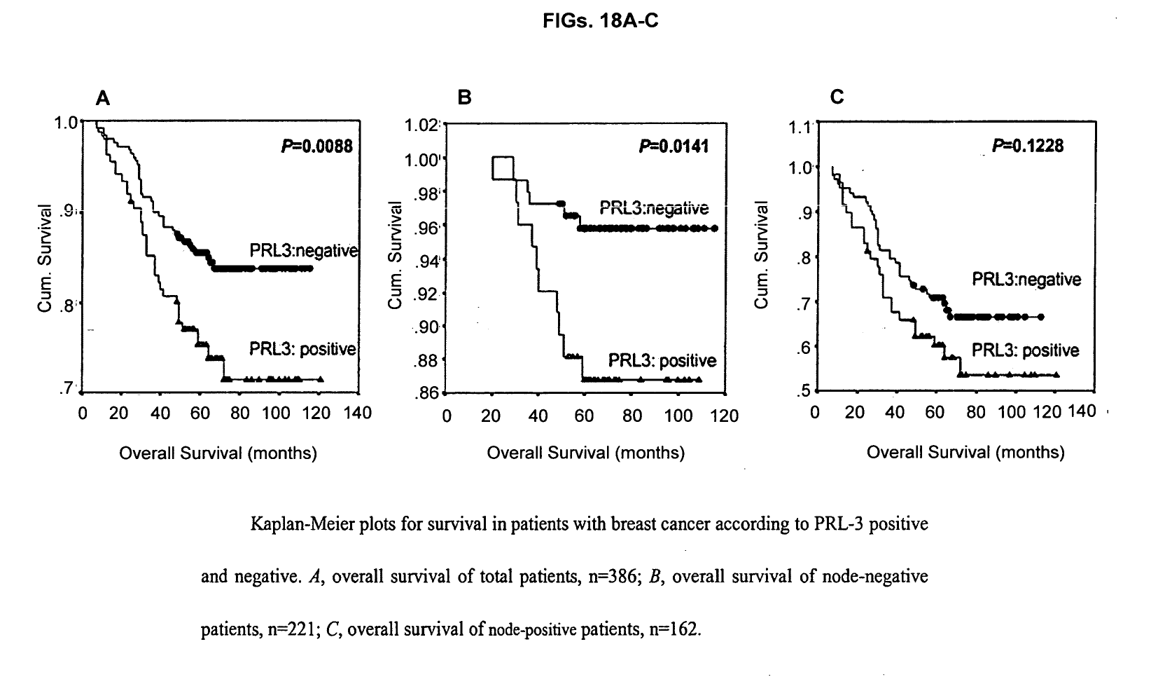

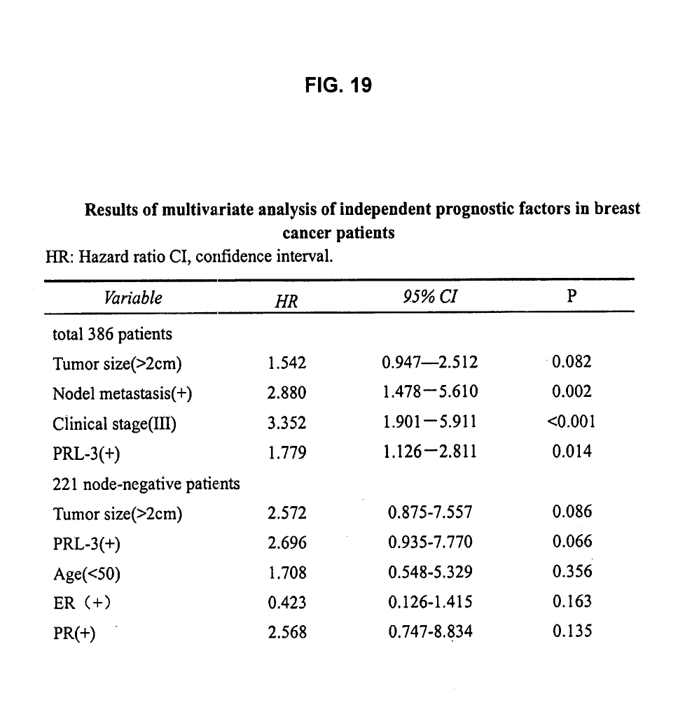

Patients (n=386) were followed over an extended period of 5 years. The 5-year overall survival rate was 80.83% in the whole population. In univariate analysis, PRL-3 expression was significantly associated with clinical outcome. Patients with a high PRL-3 expression level exhibited a lower 5-year OS rate than those with a low level of PRL-3 expression (73.8% vs 85%, P=0.009, FIG. 17 and FIG. 18A). As expected, auxiliary lymph node status (P<0.0001), tumor size (P<0.0001), and clinical stage (P<0.0001) were significantly associated with clinical outcome (see FIG. 17), whereas, patient age, ER or PR status were not significantly associated with OS in this cohort (see FIG. 17). The multivariate analysis showed that PRL-3 expression was an independent prognostic marker with regard to cancer-related survival (HR=1.8, P=0.014, FIG. 19). Although the impact of PRL-3 was less evident than a late clinical stage III (HR=3.352) or a positive lymph node status (HR=2.880), the hazard ratio for PRL-3 was 1.779. The risk of patients whose tumors were PRL-3-positive of dying from the disease within a specific time frame was 1.8 times higher than the risk of those patients whose tumors were PRL-3-negative (see FIG. 19). On the basis of multivariate Cox regression analysis, tumor size was not found to be an independent prognostic factor for OS.

### PRL-3 Expression and Survival in Node-Positive or -Negative Breast Cancer Patients

For further analysis, patients were divided into two groups by their nodal status (node-negative or node-positive). In a subgroup of patients with node-negative disease, PRL-3 expression was significantly associated with overall survival. Thus, patients with a low level of PRL-3 had a 5-year OS of 95.8% compared with a 5-year OS of 86.7% among patients with a high level of PRL-3 (P=0.014, FIG. 18B). Among the 221 patients with node-negative disease, 10 out of 76 patients with a high expression level of PRL-3 died of cancer-related causes, as did only 6 out of 145 patients with a low level of PRL-3. In multivariate analysis, patients with a high level of PRL-3 had a 2.7-fold increase in the hazard of death (95% CI 0.94-7.8, P=0.066) after adjusting for age, tumor size, ER or PR status (see FIG. 19). On the other hand, there was no significant association between PRL-3 expression and overall survival among patients with node-positive disease (P=0.123, FIG. 18C).

### Discussion

The expression of PRL-3 has been reported to promote invasive growth and metastasis of tumor cells and has been found to be an unfavorable prognostic marker. It has also been shown that suitable expression of wild-type active PRL-3 dramatically enhanced Chinese hamster ovary (CHO) cell motility and migration, whereas a catalytically inactive PRL-3 (C104S) mutant greatly reduced the effect on promoting CHO cell migration (Zeng Q., et al., Cancer Res 63:2716-2722, 2003. Mouse melanoma cells, B16F10, stably expressing PRL-3 displayed a fibroblast-like appearance and showed a much higher migratory ability than their parental cell line. PRL-3 may also facilitate lung and liver metastasis of B16F10 cells in an animal model (Wu X P, et al., Am J Pathol 164:2039-54, 2004). Another study reported that PRL-3 was the only gene consistently overexpressed in 100% of 18 colorectal cancer liver metastases examined (Saha S., et al., Science 294(5545):1343-6, 2001). Bardelli et al. found that PRL-3 mRNA expression was elevated in nearly all metastatic lesions derived from colorectal cancers, regardless of the metastatic sites (Bardelli A., et al., Clin Cancer Res 9(15):5607-15, 2003).

This study provided evidence that supports a causal role of PRL-3 in breast tumor metastasis. Immunohistochemistry with PRL-3-specific monoclonal antibody 3B6, demonstrated that PRL-3 expression was significantly associated with clinical outcome, as patients with PRL-3-negative tumors had substantially longer OS than did patients with PRL-3-positive tumors (P=0.009). Furthermore, multivariate analysis showed that positive PRL-3 expression was an independent marker for OS after adjusting for other prognostic factors. These findings supported the notion that PRL-3 may play an important role in breast cancer progression. This was consistent with previous findings that associated PRL-3 expression with decreased survival of a subset of colorectal cancer patients (see FIG. 12). In addition, PRL-3 was found to have a role in increasing tumor cell metastatic ability in colorectal cancer and other tumor types, such as in gastric and ovarian cancer (Kato H., et al., Clin Cancer Res 10(21):7318-28, 2004; Miskad U. A., et al., Pathobiology 71(4):176-84, 2004; Polato F., et al., Clin Cancer Res 11(19): 6835-6839, 2005).

In the present study, PRL-3 expression was observed to be neither associated with the tumor size nor with auxiliary lymph node status, suggesting that PRL-3 expression was independent of lymph node metastases. Indeed, PRL-3 expression was not associated with clinical outcome in patients with node-positive disease, but very importantly, it was clearly linked to a poor clinical outcome in patients with node-negative disease. As approximately 90% of node-negative breast cancer patients eventually succumb to the disease due to distant metastases, the study raised the hypothesis that PRL-3 may contribute to breast cancer metastasis, particularly in node-negative patients. The results also indicated that PRL-3 expression may be an earlier molecular event for breast cancer metastasis.

PRL-3 protein was predominantly located in the cytoplasm and cytoplasmic membrane. The distribution pattern of PRL-3 in the cell membranes may correlate with the metastatic ability of the tumor cells. A previous study showed that cells expressing PRL-3 were enriched in several membrane processes including protrusions, ruffles and some vacuolar-like membrane extension, processes which have been reported to play a role in invasion and cell movement (Small J. V., et al., Trends Cell Biol 12:112-120, 2002; Nobes C. D. and Hall A., J Cell Biol 144: 1235-1244, 1999). PRL-3 may induce dephosphorylation of target substrates at the cell membrane and modulate the organization of the plasma membrane in such a way as to promote cell motility and loss of adhesion (Matter W. F. et al., Biochem Biophys Res Commun 283:1061-1068, 2001). Experiments have shown that PRL-3 may activate the mitogen-associated protein kinase pathway mediated by association with a protein located at the membrane which is involved in cell migration and invasion (data not shown).

Lymph node-negative patients presently account for almost two-thirds of all breast cancers patients. Nearly 70% of those patients could represent a long-term survival cohort, even without adjuvant therapy; however, clinicians are not able to precisely discriminate those women who are without detectable auxiliary metastases but will develop metastatic disease from those who may be completely cured. Currently, the main prognostic markers in node-negative breast cancer are age, tumor size, histological grade, and ER or PR status. Although these markers may provide some useful information in clinical practice, in general, their predictive value is limited. Therefore, searching for precise markers that predict the clinical course of node-negative patients remains a clinical challenge. A great effort has been made in recent years to identify the proportion of patients who are at high risk for breast cancer recurrence. Several previous studies have attempted to determine the value of genetic alterations as prognostic markers for postoperative node-negative breast cancer patients. These include amplification of the HER-2/erbB2 gene (Michael F. P., et al., J Clin Oncol 15:2894-2904, 1997), mutations of p53 (Barry I., et al., Clin Cancer Res 4:1597-1602, 1998), expression level of extracellular matrix components (Suwiwat S., et al., Clin Cancer Res 10 (7):2491-2498, 2004) and cyclin E (Keyomarsi K., et al., N Engl J Med 347(20):1566-75, 2002). This study has demonstrated that PRL-3 is a strong prognostic marker among node-negative patients. More importantly, the predictive role of PRL-3 was found to be substantially stronger than that of age, tumor size, ER or PR status in this subgroup. In contrast PRL-3 seemed to play a limited predictive role in node-positive patients.

In conclusion, the study suggested that PRL-3 expression could serve as an independent prognostic factor in breast cancer patients, particularly for node-negative patients. Detection of PRL-3 expression may provide useful information to discriminate between the node-negative patients who may have a high risk of relapse, and spare the low risk of patients from having to undergo harmful chemotherapy.

While this invention has been particularly shown and described with references to preferred embodiments thereof, it will be understood by those skilled in the art that various changes in form and details may be made therein without departing from the scope of the invention encompassed by the appended claims.

1. An antibody which specifically binds to a protein tyrosine phosphatase type IVA, member 3 (PRL-3) polypeptide wherein said antibody does not bind PRL-1 or PRL-2 and provided that said antibody does not bind to amino acid residues 162 through 173 of the PRL-3 polypeptide.

2. A monoclonal antibody which specifically binds a PRL-3 polypeptide wherein said antibody does not bind PRL-1 or PRL-2.

3. The monoclonal antibody of claim 2 wherein said antibody is 3B6 or 5D3.

4. The monoclonal antibody of claim 3 wherein said antibody is produced by a hybridoma having CMGCC No. 1197 or CMGCC No. 1302.

5-8. (canceled)

9. A method of detecting the presence or absence of PRL-3, or a portion thereof, in a biological sample obtained from a subject comprising:
a.) contacting the biological sample to be tested with an antibody or antigen-binding fragment thereof, which specifically binds PRL-3, or a portion thereof, under conditions suitable for binding of the antibody to PRL-3, or a portion thereof, wherein said antibody binds a PRL-3 polypeptide, thereby forming an antibody-PRL-3 complex; and
b.) detecting the presence of the antibody-PRL-3 complex in the sample,
wherein detection of the complex, indicates the presence of PRL-3 in the sample.

10. The method of claim 9 wherein the presence of PRL-3 in the sample predicts the development of or indicates the existence of breast, colorectal or gastric cancer metastases in the subject.

11. The method of claim 9 wherein the presence of PRL-3 in the sample indicates the existence of invasive carcinoma cells.

12. The method of claim 9 wherein the presence of PRL-3 in the sample indicates the existence of tumor cell vasculature in the subject.

13. The method of claim 9 wherein said antibody is detectably labeled.

14. The method of claim 9 further comprising adding an agent which is detectably labeled such that the agent specifically binds to said antibody.

15. The method of claim 9 wherein the antibody is a monoclonal antibody.

16. The method of claim 15 wherein said monoclonal antibody is selected from the group consisting of 3B6 and 5D3.

17. The method of claim 9 wherein the sample to be tested is a biopsy tissue sample, blood, serum, saliva, urine, cerebral spinal fluid, cell lysate or a stool sample.

18. A method of detecting tumor cells in a biological sample obtained from a mammalian subject comprising:
a.) contacting the biological sample to be tested with an antibody or antigen-binding fragment thereof, which specifically binds PRL-3, or a portion thereof, under conditions suitable for binding of the antibody to PRL-3, or a portion thereof, wherein said antibody binds a PRL-3 polypeptide, thereby forming an antibody-PRL-3 complex; and
b.) detecting the presence of the antibody-PRL-3 complex in the sample,
wherein detection of the complex indicates the presence of PRL-3 in the sample.

19. The method of claim 18 wherein the presence of PRL-3 in the sample predicts the development of or indicates the existence of breast, colorectal or gastric cancer metastases in the subject.

20. The method of claim 18 wherein the presence of PRL-3 in the sample indicates the existence of invasive carcinoma cells.

21. The method of claim 18 wherein the presence of PRL-3 in the sample indicates the existence of tumor cell vasculature in the subject.

22. The method of claim 18 wherein said antibody is detectably labeled.

23. The method of claim 18 further comprising adding an agent which is detectably labeled such that the agent specifically binds to said antibody.

24. The method of claim 18 wherein the antibody is a monoclonal antibody.

25. The method of claim 24 wherein said monoclonal antibody is selected from the group consisting of 3B6 and 5D3.

26. The method of claim 18 wherein the sample to be tested is a biopsy tissue sample, blood, serum, saliva, urine, cerebral spinal fluid, cell lysate or a stool sample.

27. The method of claim 18 wherein the subject is a human.

28. A method of detecting cancer progression comprising:
a.) contacting a biological sample to be tested with an antibody or antigen-binding fragment thereof, which specifically binds PRL-3, or a portion thereof, under conditions suitable for binding of the antibody to PRL-3, or a portion thereof, wherein said antibody binds a PRL-3 polypeptide, thereby forming an antibody-PRL-3 complex; and
b.) detecting the presence of the antibody-PRL-3 complex in the sample,
wherein detection of the antibody-PRL-3 complex in the sample indicates cancer progression.

29. The method of claim 28 wherein cancer progression is selected from the group consisting of: an advance in tumor stage or cancer metastases, invasion or angiogenesis.

30. The method of claim 28 wherein said antibody is detectably labeled.

31. The method of claim 28 further comprising adding an agent which is detectably labeled such that the agent specifically binds to said antibody.

32. The method of claim 28 wherein said antibody is a monoclonal antibody selected from the group consisting of mAb 3B6 and 5D3.

33. The method of claim 28 wherein the sample to be tested is a tumor tissue sample, blood, serum, saliva, urine, cerebral spinal fluid, cell lysate or a stool sample.

34. A method of detecting the metastatic progression of gastric, breast or colon tumor cells to distal organs in a mammalian subject with gastric, breast or colorectal cancer comprising:
a.) administering to the subject an antibody which specifically binds PRL-3, or a portion thereof, under conditions suitable for binding of the antibody to PRL-3, or a portion thereof; and
b.) detecting the presence of the antibody-PRL-3 complex in one or more organs,
wherein the presence of an antibody-PRL-3 complex in distal organs indicates the metastatic progression of the colon, breast or gastric tumor cells.

35. The method of claim 34 wherein said distal organs are selected from the group consisting of lymph nodes, liver, lung, peritoneum, brain, bone and ovaries.

36. A method for determining the prognosis of survival of a mammalian subject with colon, gastric or breast cancer by analyzing a biological test sample obtained from the subject comprising:
a.) contacting the test sample from the subject with an antibody which specifically binds PRL-3, or a portion thereof, under conditions suitable for binding of the antibody to PRL-3, wherein the antibody binds to PRL-3, thereby forming an antibody-PRL-3 complex,
b.) detecting the antibody-PRL-3 complex in the test sample; and
c.) comparing the level of the antibody-PRL-3 complex in the test sample with the level of the antibody-PRL-3 complex in a suitable control sample,
wherein the level of the antibody-PRL-3 complex is indicative of the level of PRL-3 expression, and the level of PRL-3 expression at, or above a predetermined threshold, is indicative of the prognosis of the subject's survival.

37. The method of claim 36 wherein the antibody is a monoclonal antibody selected from a group consisting of 3B6 and 5D3.

38. The method of claim 36 wherein said antibody is detectably labeled.

39. The method of claim 36 further comprising combining an agent which is detectably labeled such that the agent specifically binds to said antibody.

40. The method of claim 36 wherein the biological sample is a tissue sample and the analysis is by immunohistochemistry, wherein the level of PRL-3 expression is determined by the staining intensity or subcellular localization of the antibody-PRL-3 complex.

41. The method of claim 36 wherein the subcellular localization of the antibody-PRL-3 complex to the cytoplasmic membrane indicates a decreased survival time for the mammalian subject.

42. The method of claim 36 wherein expression levels of PRL-3 at, or higher than a predetermined threshold indicate a decreased survival time of said mammalian subject.

43. The method of claim 36 wherein expression levels of PRL-3 at, or higher than a predetermined threshold are a prognostic marker for late stage cancer.

44. The method of claim 36 wherein said mammal is a human.

45. A method of preventing metastases in a mammalian subject diagnosed with cancer comprising administering to the mammal an antibody which specifically binds PRL-3, or a portion thereof, whereby said antibody binds to PRL-3, or a portion thereof, thereby inactivating the metastatic activity of PRL-3, thereby preventing the metastases of the cancer.

46. The method of claim 45 wherein the cancer is gastric, colorectal or breast cancer.

47. The method of claim 45 wherein the antibody is administered in combination with other therapies.

48. The method of claim 45 wherein the antibody is a monoclonal antibody selected from a group consisting of mAb 3B6 and 5D3.

49. A method for treating a mammalian patient with metastatic or invasive cancer comprising administering to the patient an effective amount of an antibody which specifically binds PRL-3, or a portion thereof, whereby the antibody binds a PRL-3 expressing cell and causes the death of the cell.

50. The method of claim 49 wherein the metastatic cancer is breast, gastric or colorectal cancer and the invasive cancer is gastric or breast carcinoma.

51. The method of claim 49 wherein the antibody is cytolytic.

52. The method of claim 49 wherein the antibody is conjugated to a cytotoxic or chemotherapeutic agent, a toxin or a radionucleotide.

53. The method of claim 49 wherein the antibody is administered as an adjuvant therapy in multiple doses.

54. The method of claim 49 wherein the antibody is administered in combination with other therapies.

55. The method or claim 49 wherein the antibody is selected from a group consisting of mAb 3B6 and 5D3.

56-71. (canceled)


SEQUENCE LISTING (16 sequences)

SEQ ID NO: 1 - protein, 173 aa, homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 2 - DNA, 1396 bp, homo sapiens
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccgggctccagtccttgtggcgctggcgcttattgagagcgggatgaagtacgaggacgccatccagttcatccgccagaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggacccgtgaaaaaaaaaaaaaaaaaaaaaaaaaaaaaaaaaaaaaaaaaaaaaaaaaaaaaaaaaaaaaaaa

SEQ ID NO: 3 - protein, 173 aa, homo sapiens
MARMNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 4 - protein, 167 aa, homo sapiens
MNRPAPVEISYENMRFLITHNPTNATLNKFTEELKKYGVTTLVRVCDATYDKAPVEKEGIHVLDWPFDDGAPPPNQIVDDWLNLLKTKFREEPGCCVAVHCVAGLGRAPVLVALALIECGMKYEDAVQFIRQKRRGAFNSKQLLYLEKYRPKMRLRFRDTNGHCCVQ

SEQ ID NO: 5 - protein, 173 aa, mus musculus
MARMNRPAPVEVSYRHMRFLITHNPSNATLSTFIEDLKKYGATTVVRVCEVTYDKTPLEKDGITVVDWPFDDGAPPPGKVVEDWLSLLKAKFYNDPGSCVAVHCVAGLGRAPVLVALALIESGMKYEDAIQFIRQKRRGAINSKQLTYLEKYRPKQRLRFKDPHTHKTRCCVM

SEQ ID NO: 6 - DNA, 20 bp, Artificial Sequence
  Primer
gtagacttcagtttctttgc

SEQ ID NO: 7 - DNA, 27 bp, Artificial Sequence
  Primer
atcggatccatgaaccgcccagctcct

SEQ ID NO: 8 - DNA, 20 bp, Artificial Sequence
  Primer
caagttccacttccagtagc

SEQ ID NO: 9 - DNA, 27 bp, Artificial Sequence
  Primer
catgaattcttattgaatgcaacagtt

SEQ ID NO: 10 - DNA, 20 bp, Artificial Sequence
  Primer
actttccccatcacactcac

SEQ ID NO: 11 - DNA, 27 bp, Artificial Sequence
  Primer
atcggatccatgaaccgtccagcccct

SEQ ID NO: 12 - DNA, 21 bp, Artificial Sequence
  Primer
cctctaaatggcacaatcaag

SEQ ID NO: 13 - DNA, 28 bp, Artificial Sequence
  Primer
catgaattcctactgaacacagcaatgc

SEQ ID NO: 14 - DNA, 27 bp, Artificial Sequence
  Primer
atcggatccatggctcggatgaaccgc

SEQ ID NO: 15 - DNA, 17 bp, Artificial Sequence
  Primer
tgacgaccaggcccagc

SEQ ID NO: 16 - DNA, 27 bp, Artificial Sequence
  Primer
catgaattcctacataacgcagcaccg